(19) United States
(12) Patent Application Publication
(10) Pub. No.: US 2004/0086508 A1
(43) Pub. Date: May 6, 2004

(54) Treatment of organ transplant rejection

(75) Inventors: Boris Skurkovich, Pawtucket, RI (US); Simon Skurkovich, Rockville, MD (US)

Correspondence Address: MORGAN, LEWIS & BOCKIUS LLP, 1701 MARKET STREET, PHILADELPHIA, PA, 19103-2921, US

(73) Assignee: Advanced Biotherapy, Inc.

(21) Appl. No.: 10/631,439

(22) Filed: Jul. 30, 2003

Related U.S. Application Data
(63) Continuation-in-part of application No. 10/372,644, filed on Feb. 21, 2003.
Application No. 10/372,644 is a continuation of application No. 09/894,287, filed on Jun. 28, 2001, now Pat. No. 6,534,059.
(60) Provisional application No. 60/295,895, filed on Jun. 5, 2001.

(51) Int. Cl. A61K 39/395

(52) U.S. Cl. 424/145.1

(57) ABSTRACT

The present invention comprises and utilizes methods and compositions for treating organ and cell transplant rejection. Compositions comprising antibodies to gamma interferon and tumor necrosis factor alone, together, and in combination with other drugs are described. Further disclosed are methods of treating organ transplant rejection comprising the administration of antibodies to cytokine receptors, soluble receptors, and extracorporeal removal of cytokines and other autoimmune mediators.

### CROSS-REFERENCE TO RELATED APPLICATIONS

The present application is a-continuation-in-part of co-pending U.S. application Ser. No. 10/372,644, filed Feb. 21, 2003, which is a continuation of U.S. application Ser. No. 09/894,287, filed Jun. 28, 2001, now issued as U.S. Pat. No. 6,534,059, which is entitled to priority under 35 U.S.C. §119(e) to U.S. Provision/al Application No. 60/295,895, filed Jun. 5, 2001, all of which are hereby incorporated by reference in their entirety herein.

### BACKGROUND OF THE INVENTION

The ability of the mammalian immune system to recognize “self” versus “non-self” antigens is vital to successful host defense against invading microorganisms. “Self” antigens are those which are not detectably different from an animal's own constituents, whereas “non-self” antigens are those which are detectably different from or foreign to the mammal's constituents. A normal mammalian immune system functions to recognize “non-self antigens” and attack and destroy them. An autoimmune disorder such as for example, rheumatoid arthritis, insulin-independent diabetes mellitus, acquired immune deficiency syndrome (AIDS), multiple sclerosis, and the like, results when the immune system identifies “self” antigens as “non-self”, thereby initiating an immune response against the mammal's own body components (i.e., organs and/or tissues). This creates damage to the mammal's organs and/or tissues and can result in serious illness or death.

Predisposition of a mammal to an autoimmune disease is largely genetic; however, exogenous factors such as viruses, bacteria, or chemical agents may also play a, role. Autoimmunity can also surface in tissues that are not normally exposed to lymphocytes such as for example, neural tissue and the eye (particularly the lens or the cornea). When a tissue not normally exposed to lymphocytes becomes exposed to these cells, the lymphocytes may recognize the surface antigens of these tissues as “non-self” and an immune response may ensue. Autoimmunity may also develop as a result of the introduction into the animal of antigens which are sensitive to the host's self antigens. An antigen which is similar to or cross-reactive with an antigen in an mammal's own tissue may cause lymphocytes to recognize and destroy both “self” and “non-self” antigens.

It has been suggested that the pathogenesis of autoimmune diseases is associated with a disruption in synthesis of interferons and other cytokines often induced by interferons (Skurkovich et al., Nature 217:551-552, 1974; Skurkovich et al., Annals of Allergy, 35:356, 1975; Skurkovich et al., J. Interferon Res. 12, Suppl. 1:S110, 1992; Skurkovich et al., Med. Hypoth., 41:177-185, 1993; Skurkovich et al., Med. Hypoth., 42:27-35, 1994; Gringeri et al., Cell. Mol. Biol. 41(3):381-387, 1995; Gringeri et al., J. Acquir. Immun. Defic. Syndr., 13:55-67, 1996). In particular, interferon (IFN) gamma plays a significant pathogenic role in autoimmune dysfunction. IFN gamma stimulates cells to produce elevated levels of HLA class II antigens (Feldman et al., 1987, “Interferons and Autoimimunity”, In: IFN γ, p. 75, Academic Press). It is known that IFN gamma participates in the production of tumor necrosis factor (TNF), and it is also known that TNF also plays a role in stimulation of production of autoantibodies. In view of this, therapies to modulate these cytokines have been developed. Clinical success in treating several autoimmune diseases using antibodies to IFN gamma has been reported (Skurkovich et al., U.S. Pat. No. 5,888,511).

However, while an autoimmune response is considered to be typical in diseases such as multiple sclerosis and rheumatoid arthritis, one area of medicine where treatment of autoimmune or hyperimmune responses has not been fully explored is the area of transplant therapy. Autoimmunity arising from transplant rejection is typical in transplant patients. Rejection of a transplant is the organism's normal reaction to invading foreign antigens. In particular, transplantation of tissues or organs such as the eye, which is not normally exposed to lymphocytes, skin, heart, kidney, liver, bone marrow, and other organs, have a high rate of rejection, which rejection is largely the result of a hyperimmune reaction.

There are approximately 82,000 people waiting for an organ transplant in the United States at any one time. Of these, about 56,000 are waiting for a kidney transplant, about 1,500 are waiting for a pancreas (beta islet cells) transplant, about 17,000 are waiting for a liver transplant, and about 4,000 are waiting for a heart transplant. Approximately 2000 organ transplants are performed each month. The number of candidate recipients far outweighs the number of available organs.

After the organ has been transplanted into the patient and the patient's immune system is suppressed to prevent rejection of the new organ, two common outcomes frequently result. The first is infection. If detected early, most infections can be treated with antibiotics or other standard therapies along with supportive treatment with few or no long term effects to the patient or transplanted organ. The second occurrence is rejection.

Organ transplant rejection is traditionally based on histopathologic observances, rather than immune effector mechanisms, and comprises three separate categories. Hyperacute rejection is characterized by rapid thrombotic occlusion of the graft vasculature within minutes to hours after organ transplantation. Hyperacute rejection is mediated in large part by pre-existing antibodies that bind to the epithelium and activate the complement cascade. Complement activation results in endothelial cell damage and subsequent exposure of the basement membrane, resulting in the activation of platelets, leading to thrombosis and vascular occlusion. Hyperacute rejection has become less common due to blood antigen and MHC molecule matching between the donor organ and the recipient.

Acute rejection is sub-classified into acute vascular rejection and acute cellular rejection. Acute vascular rejection is characterized by necrosis of individual cells in the graft blood vessels. The process is similar to that of hyperacute rejection, but onset is often slower, within one week of rejection, and a T cell component may be involved. Acute vascular rejection is initiated by a response to alloantigens present on the vascular endothelial cells of the donor organ, resulting in the release of a cytokine cascade, inflammation, and eventual necrosis. Acute cellular rejection is often characterized by necrosis of the essential or parenchymal cells of the transplanted organ caused by the infiltration of host T lymphocytes and macrophages. The lymphocytes involved are usually cytotoxic T lymphocytes (CTL) and macrophages, both resulting in lysis of targeted cells. The CTLs are usually specific for graft alloantigens displayed in the context of MHC class I molecules.

Chronic rejection is the major cause of allograft loss and is characterized by fibrosis and loss of normal organ structures. Fibrosis may be the result of wound healing following the cellular necrosis of acute rejection, or may occur independently and without prior acute rejection. In addition, chronic rejection may lead to vascular occlusions thought to stem from a delayed type hypersensitivity response to alloantigens present on the transplanted organ. These alloantigens stimulate lymphocytes to secrete cytokines which attract macrophages and other effector cells eventually leading to an arteriosclerosis-like blockage.

Organ transplant rejection is currently prevented by matching the donor organ with the recipient and by immunosuppressive therapy after the transplant procedure is completed. Matching the donor organ to the recipient comprises matching the ABO blood antigens and the HLA molecules. Three or four out of a possible four HLA-A and HLA-B correlates with better graft survival, especially after the first year of transplantation. HLA-DR and—DQ matching also correlates with survival of the transplanted organ.

After the donor organ has been matched and implanted, organ transplant recipients must take immunosuppressive medication for the rest of their lives. Initial doses of immunosuppressive medications are usually quite high, and are accompanied by varied and serious side effects. Over time, the doses of immunosuppressive medication can be lowered with an accompanying decrease in side effects and infection susceptibility. However, the generalized immunosuppressive medications currently in use are not without significant problems. In fact, the general immunosuppressants currently used suppress all immunological reactions to antigens, either those present on the transplanted organ or those encountered from infectious microbes, tumor cells, or, other disease causing anomalies, and overwhelming infection remains the leading cause of death in transplant recipients.

Corticosteroids, such as prednisolone, are usually administered in high doses (2 to 20 mg/kg) immediately following transplantation, and are then reduced to a maintenance dose indefinitely (0.2 mg/kg/day). Despite adverse side effects such as weight gain, high-blood pressure, facial puffiness, muscle weakness, growth retardation in children, and occasional psychological disturbances, prednisolone and other corticosteroids are often administered to transplant recipients for the rest of their lives. Even with the co-administration of other immunosuppressants, the chance of organ rejection increases when prednisolone administration ceases.

Azathioprine is an anti-metabolite usually administered at the time of transplantation via oral or intravenous routes (1 to 2.5 mg/kg/day). The primary toxic side effect of azathioprine is bone marrow depression, which increases susceptibility to infection. Cases of hepatitis resulting from azathioprine administration have also been reported. Azathioprine is often administered in conjunction with cyclosporine.

Cyclophosphamide is an alkylating agent used in place of azathioprine. However, severe side effects are common, and often include hemorrhagic cystitis, infertility and alopecia. Both azathioprine and cyclophosphamide act by inhibiting the maturation of lymphocytes from precursor cells, and kill proliferating mature T cells that have been stimulated by donor organ and other antigens. Other rapidly proliferating cells, such as those of the gut lining, are also targeted by azathioprine and cyclophosphamide.

Cyclosporine (NEORAL, GENGRAF, SANDIMMUNE) is often credited with making organ transplants a viable reality, and is administered at initial doses of about 6 to 12 mg/kg/day, and reduced to a maintenance level of about 3 to 5 mg/kg/day. Cyclosporine, or cyclosporin A, is a cyclic peptide derived from a species of fungus. Cyclosporine spares the bone marrow and acts directly on T cells to inhibit transcription of certain genes, including IL-2, blocking the IL-2-dependent growth of T cells. While cyclosporine is widely considered as ushering in the success of organ transplantation, and increasing organ survival to five years or more, it is not without its side effects.

Nephrotoxicity, hepatotoxicity, refractory hypertension, hirsutism and neoplasia formation are all known to occur. Neoplasia formation is often associated with Epstein-Barr Virus activation in the face of reduced immune function, resulting in lymphomas and B-cell lymphoproliferative disorders. The nephrotoxic effects of cyclosporine can negate any benefit of a kidney transplant. Further, the therapeutically effective amount of cyclosporine varies from patient to patient, and there is little reliable correlation between physiological levels of cyclosporine and the toxic side effects in any one patient.

Tacrolimus (PROGRAF, FK506) is the byproduct released from the growth of a Streptomyces species used to suppress the immune system in liver transplants. Tacrolimus is usually administered at a dose of about 0.15 to 0.30 mg/kg/day orally and 0.05 to 0.1 mg/kg/day if administered intravenously. The adverse effects are similar to those of cyclosporine, and may also cause diabetes. Tacrolimus is often used where cyclosporine is too toxic or ineffective.

Other immunosuppressants are well known in the art, and include mycophenolate mofetil (CELLCEPT), sirolimus (RAPAMUNE), daclizumab (ZENAPAX) and basilecmab (SIMULECT).

Various antibodies have been used in the management of rejection following organ transplant. Antilymphocyte globulin (ALG) and antithymocyte globulin (ATG) can be used in conjunction with other immunosuppressants, allowing the administration of lower and less toxic doses. Administration of highly purified fractions of these antibodies intravenously has reduced the occurrence of anaphylactic reactions, serum sickness, and glomerulonephritis.

OKT3 is a murine monoclonal antibody that binds to the CD3 (T cell receptor) molecule, and is administered at a dose of 5 mg/day intravenously for 10 too 14 days at the time of an acute rejection episode. The non-specific activation of the bound T cells causes a cytokine release characterized by fever, myalagia, and central nervous system and gastrointestinal irritation. Following this period, OKT3 modulates the T cell reaction, and delays the onset and reduces the number of rejection occurrences. OKT3 is often used as a prophylactic agent, but incidences of a patient developing neutralizing antibodies to OKT3 and Epstein-Barr virus induced lymphoproliferation limit its use.

Irradiation is also used in limited situations in pre-transplant preparation or during organ rejection episodes. In some cases the site of transplantation and the donor organ are both irradiated. Irradiation in humans is still in the experimental stages of use.

Much scientific effort has been expended on trying to develop new methods to prevent rejection of organ transplant rejection. Progress in preventing organ transplant with anti-cytokine antibodies has thus far been limited to preventing or possibly treating GVHD in a murine model (U.S. Pat. No. 5,672,347) and preventing skin transplant rejection in non-human primates (Stevens, et al., 1990, Transplantation 50: 856-861; Jonker, et al., WO 90/10707).

Hyperimmune reactions including rejection of tissue transplants in the eye and other organs are of considerable concern. Corneal transplants, lens replacements, and the like, are frequently rejected when transplanted into a human patient. In addition, other diseases in the eye, such as for example, keratoconjunctivitis sicca (dry eye syndrome), episcleritis, scleritis, Mooren's ulcer, ocular cicatricial pemphigoid, orbital pseudotumor, iritis, central serous retinopathy, Graves' ophthalmopathy, chorioretinitis, Sjogren's syndrome, diabetic retinopathy, macular dystrophy, macular degeneration, glaucoma, and Stevens-Johnson syndrome may also be the result of a hyperimmune reaction in the eye. Systemic infections, such as tuberculosis, syphilis, AIDS, toxoplasmosis infection, and cytomegalovirus retinitis, may also cause eye diseases, including but not limited to, uveitis, enophthalmitis, retinitis, choroiditis, and retinal necrosis. These types of hyperimmune reactions typically result in blurred vision and eventually blindness. Current therapies to treat such hyperimmune responses include corticosteroid treatment, including dexamethasone, and treatment with an anti-inflammatory preparation. To date, there are no successful or long-term methods or compositions for effectively treating hyperimmune reactions in the mammalian eye and other organs. The present invention provides such methods and compositions.

### SUMMARY OF THE INVENTION

The invention includes a method of treating organ transplant rejection in a human patient, where the organ is not skin. The method comprises administering to the patient an effective amount of a combination of an antibody that specifically binds interferon gamma and an antibody that specifically binds tumor necrosis factor alpha.

In one aspect, the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

In another aspect, the antibody is administered by the route selected from the group consisting of intramuscularly, intravenously, intradermally, cutaneously, subcutaneously, rectally, ionophoretically, topically, locally, and inhalation.

In yet another aspect, the antibody is selected from the group consisting of a polyclonal antibody, a monoclonal antibody, a synthetic antibody, a heavy chain antibody and a humanized antibody.

In still another aspect, the heavy chain antibody is selected from the group consisting of a camelid antibody, a heavy chain disease antibody, and a variable heavy chain immunoglobulin.

In another aspect, the human patient is optionally administered an immunosuppressant.

The invention includes a method of treating heart transplant rejection in a human patient, the method comprising administering to the patient an effective amount of a combination of an antibody that specifically binds interferon gamma and an antibody that specifically binds tumor necrosis factor alpha.

In one aspect, the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof;

In another aspect, the antibody is administered by the route selected from the group consisting of intramuscularly, intravenously, intradermally, cutaneously, subcutaneously, rectally, ionophoretically, topically, locally, and inhalation.

In yet another aspect, the antibody is selected from the group consisting of a polyclonal antibody, a monoclonal antibody, a synthetic antibody, a heavy chain antibody and a humanized antibody.

In still another aspect, the heavy chain antibody is selected from the group consisting of a camelid antibody, a heavy chain disease antibody, and a variable heavy chain immunoglobulin.

In another aspect, the human patient is optionally administered an immunosuppressant.

The invention includes a method of treating kidney transplant rejection in a human patient, the method comprising administering to the patient an effective amount of a combination of an antibody that specifically binds interferon gamma and an antibody that specifically binds tumor necrosis factor alpha.

In one aspect, the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

In another aspect, the antibody is administered by the route selected from the group consisting of intramuscularly, intravenously, intradermally, cutaneously, subcutaneously, rectally, ionophoretically, topically, locally, and inhalation.

In yet another aspect, the antibody is selected from the group consisting of a polyclonal antibody, a monoclonal antibody, a synthetic antibody, a heavy chain antibody and a humanized antibody.

In still another aspect, the heavy chain antibody is selected from the group consisting of a camelid antibody, a heavy chain disease antibody, and a variable heavy chain immunoglobulin.

In another aspect, the human patient is optionally administered an immunosuppressant.

The invention includes a method of treating liver transplant rejection in a human patient, the method comprising administering to the patient an effective amount of a combination of an antibody that specifically binds interferon gamma and an antibody that specifically binds tumor necrosis factor alpha.

In one aspect, the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

In another aspect, the antibody is administered by the route selected from the group consisting of intramuscularly, intravenously, intradermally, cutaneously, subcutaneously, rectally, ionophoretically, topically, locally, and inhalation.

In yet another aspect; the antibody is selected from the group consisting of a polyclonal antibody, a monoclonal antibody, a synthetic antibody, a heavy chain antibody and a humanized antibody.

In still another aspect; the heavy chain antibody is selected from the group consisting of a camelid antibody, a heavy chain disease antibody, and a variable heavy chain immunoglobulin.

In another aspect, the human patient is optionally administered an immunosuppressant.

The invention includes a method of treating pancreatic beta-islet cell transplant rejection in a human patient, the method comprising administering to the patient an effective amount of a combination of an antibody that specifically binds interferon gamma and an antibody that specifically binds tumor necrosis factor alpha.

In one aspect, the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

In another aspect, the antibody is administered by the route selected from the group consisting of intramuscularly, intravenously, intradermally, cutaneously, subcutaneously, rectally, ionophoretically, topically, locally, and inhalation.

In yet another aspect, the antibody is selected from the group consisting of a polyclonal antibody, a monoclonal antibody, a synthetic antibody, a heavy chain antibody and a humanized antibody.

In still another aspect, the heavy chain antibody is selected from the group consisting of a camelid antibody, a heavy chain disease antibody, and a variable heavy chain immunoglobulin.

In another aspect, the human patient is optionally administered an immunosuppressant.

The invention includes a method of treating organ transplant rejection in a human patient, wherein the organ is not skin, the method comprising administering to the patient an effective amount of an antibody that specifically binds interferon gamma.

In one aspect, the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a:heavy chain antibody, and combinations thereof.

In another aspect, the antibody is administered by the route selected from the group consisting of intramuscularly, intravenously, intradermally, cutaneously, subcutaneously, rectally, ionophoretically, topically, locally, and inhalation.

In yet another aspect, the antibody is selected from the group consisting of a polyclonal antibody, a monoclonal antibody, a synthetic antibody, a heavy chain antibody and a humanized antibody.

In still another aspect, the, heavy chain antibody is selected from the group consisting of a camelid antibody, a heavy chain disease antibody, and a variable heavy chain immunoglobulin.

In another aspect, the human patient is optionally administered an immunosuppressant.

The invention includes a method of treating heart transplant rejection in a human patient, the method comprising administering to the patient an effective amount of an antibody that specifically binds interferon gamma.

In one aspect, the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

In another aspect, the antibody is administered by the route selected from the group consisting of intramuscularly, intravenously, intradermally, cutaneously, subcutaneously, rectally, ionophoretically, topically, locally, and inhalation.

In yet another aspect, the antibody is selected from the group consisting of a polyclonal antibody, a monoclonal antibody, a synthetic antibody, a heavy chain antibody and a humanized antibody.

In still another aspect, the heavy chain antibody is selected from the group consisting of a camelid antibody, a heavy chain disease antibody, and a variable heavy chain immunoglobulin.

In another aspect, the human patient is optionally administered an immunosuppressant.

The invention includes a method of treating kidney transplant rejection in a human patient, the method comprising administering to the patient an effective amount of an antibody that specifically binds, interferon gamma.

In one aspect, the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

In another aspect, the antibody is administered by the route selected from the group consisting of intramuscularly, intravenously, intradermally, cutaneously, subcutaneously, rectally, ionophoretically, topically, locally, and inhalation.

In yet another aspect, the antibody is selected from the group consisting of a polyclonal antibody, a monoclonal antibody, a synthetic antibody, a heavy chain antibody and a humanized antibody.

In still another aspect, the heavy chain antibody is selected from the group consisting of a camelid antibody, a heavy chain disease antibody, and a variable heavy chain immunoglobulin.

In another aspect, the human patient is optionally administered an immunosuppressant.

The invention includes a method of treating liver transplant rejection in a human patient, the method comprising administering to the patient an effective amount of an antibody that specifically binds interferon gamma:.

In one aspect, the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

In another aspect, the antibody is administered by the route selected from the group consisting of intramuscularly, intravenously, intradermally, cutaneously, subcutaneously, rectally, ionophoretically, topically, locally, and inhalation.

In yet another aspect, the antibody is selected from the group consisting of a polyclonal antibody, a monoclonal antibody, a synthetic antibody, a heavy chain antibody and a humanized antibody.

In still another aspect, the heavy chain antibody is selected from the group consisting of a camelid antibody, a heavy chain disease antibody, and a variable heavy chain immunoglobulin.

In another aspect, the human patient is optionally administered an immunosuppressant.

The invention includes a method of treating pancreatic beta-islet cell transplant rejection in a human patient, the method comprising administering to the patient an effective amount of an antibody that specifically binds interferon gamma.

In one aspect, the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

In another aspect, the antibody is administered by the route selected from the group consisting of intramuscularly, intravenously, intradermally, cutaneously, subcutaneously, rectally, ionophoretically, topically, locally, and inhalation.

In yet another aspect, the antibody is selected from the group consisting of a polyclonal antibody, a monoclonal antibody, a synthetic antibody, a heavy chain antibody and a humanized antibody.

In still another aspect, the heavy chain antibody is selected from the group consisting of a camelid antibody, a heavy chain disease antibody, and a variable heavy chain immunoglobulin.

In another aspect, the human patient is optionally administered an immunosuppressant.

The invention includes a method of treating organ transplant rejection in a human patient, wherein the organ is not skin, the method comprising administering to the patient an effective amount of an antibody that specifically binds tumor necrosis factor alpha.

In one aspect, the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof

In another aspect, the antibody is administered by the route selected from the group consisting of intramuscularly, intravenously, intradermally, cutaneously, subcutaneously, rectally, ionophoretically, topically, locally, and inhalation.

In yet another aspect, the antibody is selected from the group consisting of a polyclonal antibody, a monoclonal antibody, a synthetic antibody, a heavy chain antibody and a humanized antibody.

In still another aspect, the heavy chain antibody is selected from the group consisting of a camelid antibody, a heavy chain disease antibody, and a variable heavy chain immunoglobulin.

In another aspect, the human patient is optionally administered an immunosuppressant.

The invention includes a method of treating heart transplant rejection in a human, patient, the method comprising administering to the patient an effective amount of a n antibody that specifically binds tumor necrosis factor alpha.

In one aspect, the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment, thereof, an allelic variant thereof, a species variant thereof, a synthetic antibody, a biologically active fragment thereof, an allelic variant thereof, a species variant thereof, a heavy chain antibody, and combinations thereof.

In another aspect, the antibody is administered by the route selected from the group consisting of intramuscularly, intravenously, intradermally, cutaneously, subcutaneously, rectally, ionophoretically, topically, locally, and inhalation.

In yet another aspect, the antibody is selected from the group consisting of a polyclonal antibody, a monoclonal antibody, a synthetic antibody, a heavy chain antibody and a humanized antibody.

In still another aspect, the heavy chain antibody is selected from the group consisting of a camelid antibody, a heavy chain disease antibody, and a variable heavy chain immunoglobulin.

In another aspect, the human patient is optionally administered an immunosuppressant.

The invention includes a method, of treating kidney transplant rejection in a human patient, the method comprising administering to the patient an effective amount of an antibody that specifically binds tumor necrosis factor alpha.

In one aspect, the antibody is selected from the group consisting of a. polyclonal antibody, a biologically active fragment thereof, an allelic variant thereof, a species variant thereof, a monoclonal antibody, a biologically active fragment thereof, an allelic variant thereof a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

In another aspect, the antibody is administered by the route selected from the group consisting of intramuscularly, intravenously, intradermally, cutaneously, subcutaneously, rectally, ionophoretically, topically, locally, and inhalation. In yet another aspect, the antibody is selected from the group consisting of a polyclonal antibody, a monoclonal antibody, a synthetic antibody, a heavy chain antibody and a humanized antibody.

In still another aspect, the heavy chain antibody is selected from the group consisting of a camelid antibody, a heavy chain disease antibody, and a variable heavy chain immunoglobulin.

In another aspect, the human patient is optionally administered an immunosuppressant.

The invention includes a method of treating liver transplant rejection in a human patient, the method comprising administering to the patient an effective amount of an antibody that specifically binds tumor necrosis factor alpha.

In one aspect, the antibody is selected from, the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

In another aspect, the antibody is administered by the route selected from the group consisting of intramuscularly, intravenously, intradermally, cutaneously, subcutaneously, rectally, ionophoretically, topically, locally, and inhalation.

In yet another aspect, the antibody is selected from the group consisting of a polyclonal antibody, a monoclonal antibody, a synthetic antibody, a heavy chain antibody and a humanized antibody.

In still another, aspect, the heavy chain antibody is selected from the group consisting of a camelid antibody, a heavy chain disease antibody, and a variable heavy chain immunoglobulin.

In another aspect, the human patient is optionally administered an immunosuppressant.

The invention includes a method of treating pancreatic beta-islet cell transplant rejection in a human patient, the method comprising administering to the patient an effective amount of an antibody that specifically binds tumor necrosis factor alpha.

In one aspect, the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

In another aspect, the antibody is administered by the route selected from the group consisting of intramuscularly, intravenously, intradermally, cutaneously, subcutaneously, rectally, ionophoretically, topically, locally, and inhalation.

In yet another aspect, the antibody is selected from the group consisting of a polycldnal antibody, a monoclonal antibody, a synthetic antibody, a heavy chain antibody and a humanized antibody.

In still another aspect, the heavy chain antibody is selected from the group consisting of a camelid antibody, a heavy chain disease antibody, and a variable heavy chain immunoglobulin.

In another aspect, the human patient is optionally administered an immunosuppressant.

The invention includes a method of treating lung transplant rejection in a human patient, the method comprising administering to the patient an effective amount of a combination of an antibody that specifically binds tumor necrosis factor alpha and an antibody that specifically binds interferon gamma.

In one aspect, the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

In another aspect, the antibody is administered by the route selected from the group consisting of intramuscularly, intravenously, intradermally, cutaneously, subcutaneously, rectally, ionophoretically, topically, locally, and inhalation.

In yet another aspect, the antibody is selected from the group consisting of a polyclonal antibody, a monoclonal antibody, a synthetic antibody, a heavy chain antibody and a humanized antibody.

In still another aspect, the heavy chain antibody is selected from the group consisting of camelid antibody, a heavy chain disease antibody, and a variable heavy chain immunoglobulin.

In another aspect, the human patient is optionally administered an immunosuppressant.

The invention includes a method of treating lung transplant rejection in a human patient, the method comprising administering to the patient an effective amount of an antibody that specifically binds tumor necrosis factor alpha.

In one aspect, the antibody is 'selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof

In another aspect, the antibody is administered by the route selected from the group consisting of intramuscularly, intravenously, intradermally, cutaneously, subcutaneously, rectally, ionophoretically, topically, locally, and inhalation.

In yet another aspect, the antibody is selected from the group consisting of a polyclonal antibody, a monoclonal antibody, a synthetic antibody, a heavy chain antibody and a humanized antibody.

In still another aspect, the heavy chain antibody is selected from the group consisting of a camelid antibody, a heavy chain disease antibody, and a variable heavy chain immunoglobulin.

In another aspect, the human patient is optionally administered an immunosuppressant.

The invention includes a method of treating lung transplant rejection in a human patient, the method comprising administering to the patient an effective amount of an antibody that specifically binds interferon gamma.

In one aspect, the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

In another aspect, the antibody is administered by the route selected from the group consisting of intramuscularly, intravenously, intradermally, cutaneously, subcutaneously, rectally, ionophoretically, topically, locally, and inhalation.

In yet another aspect, the antibody is selected from the group consisting of a polyclonal antibody, a monoclonal antibody, a synthetic antibody, a heavy chain antibody and a humanized antibody.

In still another aspect, the heavy chain antibody is selected from the group consisting of a camelid antibody, a heavy chain disease antibody, and a variable heavy chain immunoglobulin.

In another aspect, the human patient is optionally administered an immunosuppressant.

The invention includes a method of treating bone marrow transplant rejection in a human patient, the method comprising administering to the patient an effective amount of a combination of an antibody that specifically binds tumor necrosis factor alpha and an antibody that specifically binds interferon gamma.

In one aspect, the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

In another aspect, the antibody is administered by the route selected from the group consisting of intramuscularly, intravenously, intradermally, cutaneously, subcutaneously, rectally, ionophoretically, topically, locally, and inhalation.

In yet another aspect, the antibody is selected from the group consisting of a polyclonal antibody, a monoclonal antibody, a synthetic antibody, a heavy chain antibody and a humanized antibody.

In still another aspect, the heavy chain antibody is selected from the group consisting of a camelid antibody, a heavy chain disease antibody, and a variable heavy chain immunoglobulin.

In another aspect, the human patient is optionally administered an immunosuppressant.

The invention includes a method of treating bone marrow transplant rejection in a human patient, the method comprising administering to the patient an effective amount of an antibody that specifically binds tumor necrosis factor alpha.

In one aspect, the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

In another aspect, the antibody is administered by the route selected from the group consisting of intramuscularly, intravenously, intradermally, cutaneously, subcutaneously, rectally, ionophoretically, topically, locally, and inhalation.

In yet another aspect, the antibody is selected from the group consisting of a polyclonal antibody, a monoclonal antibody, a synthetic antibody, a heavy chain antibody and a humanized antibody.

In still another aspect, the heavy chain antibody is selected from the group consisting of a camelid antibody, a heavy chain disease antibody, and a variable heavy chain immunoglobulin.

In another aspect, the human patient is optionally administered an immunosuppressant.

The invention includes a method of treating bone marrow transplant rejection in a human patient, the method comprising administering to the patient an effective amount of an antibody that specifically binds interferon gamma.

In one aspect, the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

In another aspect, the antibody is administered by the route selected from the group consisting of intramuscularly, intravenously, intradermally, cutaneously, subcutaneously, rectally, ionophoretically, topically, locally, and inhalation.

In yet another aspect, the antibody is selected from the group consisting of a polyclonal antibody, a monoclonal antibody, a synthetic antibody, a heavy chain antibody and a humanized antibody.

In still another aspect, the heavy chain, antibody is selected from the group consisting of a camelid antibody, a heavy chain disease antibody, and a variable heavy chain immunoglobulin.

In another aspect, the human patient is optionally administered an immunosuppressant.

### DETAILED DESCRIPTION OF THE INVENTION

The invention includes a method of treating an organ transplant rejection in a human patient. That is, the present invention is based in part on the discovery that administration of an effective amount of an antibody that specifically binds a cytokine, including a cytokine such as interferon gamma (IFN gamma) and tumor necrosis factor alpha (TNF alpha), alone or in combination, is useful in alleviating, treating, or eliminating the rejection of a transplanted organ. The present invention encompasses treatment of organ transplant rejection when the organ is rejected by host immune mechanisms, or host versus graft disease (HVGD), as opposed to the dissimilar processes involved in graft versus host disease (GVHD). That is, the invention should not be construed to encompass methods of treating GVHD. Organ transplant rejection includes hyperacute rejection, acute rejection, and chronic rejection of organs and transplanted organs such as heart, kidney, liver, pancreas, heart-lung transplants, pancreatic beta cells (islet cells), spleen, lung, eyes, testicles, connective tissues, bone marrow, ovaries, adrenal gland, lymph glands, nerves, components of the central nervous system including the spinal cord and neural cells, hypophysis, thyroid, pituitary, hypothalamus, inner ear, bone, muscles, tendons, and other organs to be transplanted in the future.

The invention also comprises and utilizes the discovery that administration of antibodies to interferon (IFN) gamma to an animal having an autoimmune reaction in the eye is useful in alleviating or eliminating the autoimmune reaction. Such autoimmune reactions in the eye may occur as a result of transplants of eye tissue and eye diseases, including but not limited to Sjogren's syndrome, multiple sclerosis, sarcoidosis, ankylosing spondylitis, keratoconjunctivitis sicca (dry eye syndrome), episcleritis, scleritis, Mooren's ulcer, ocular cicatricial pemphigoid, orbital pseudotumor, iritis, central serous retinopathy, Graves' ophthalmopathy, chorioretinitis, Stevens-Johnson syndrome, uveitis, enophthalmitis, retinitis, choroiditis, and retinal necrosis. Autoimmune reactions in the eye may also occur as a result of contracting an infectious disease, including, but not limited to AIDS, syphilis, toxoplasmosis infection, and tuberculosis. Autoimimunity may also occur as a result of transplantation of tissue into the eye.

It is immediately apparent from the Examples disclosed herein that antibodies to IFN gamma are also useful for treatment of eye diseases which are characterized by hemorrhage and exudate collection in the eye. Hemorrhage and/or exudate may collect in the anterior chamber of the eye and is a characteristic result of an inflammatory reaction. Typically, these symptoms occur during transplant rejection (i.e., a hyperimmune response). However, the invention should not be construed as being limited solely to the examples provided-herein, as other autoimmune diseases of the mammalian eye which are at present unknown, once known, may also be treatable using the methods of the invention.

The invention includes a method of treating an eye disease characterized by a hyperimmune response in the eye of a mammal.; Briefly, the method comprises applying antibodies to gamma interferon directly to the affected eye. The method can be used to treat an autoimmune eye disease in any mammal; however, preferably, the mammal is a human.

The invention also encompasses a method of treating rejection of an organ transplant; Briefly, the method comprises administering antibodies to a human patient before, during, or after an organ transplant procedure, or at any time when an organ is being rejected by the recipient's body.

The invention also includes a method of treating rejection of an organ transplant where the organ that has been transplanted is a xenograft. As is well known in the art, xenografts are organs transplanted from one species of animal to another, different species. The organs from non-human primates and pigs are most often used for xenotransplantation. The skilled artisan will readily appreciate that xenotransplantation can diminish the current shortage of organs available for transplantation, when coupled with methods to alleviate the autoimmune rejection of a xenotransplanted organ. As an example, the presence of Gal-alpha-1,3-Gal epitopes present on the pig vascular endothelium can lead to autoimmune rejection of pig heart xenografts. The present invention therefore also comprises a method for treating organ xenograft transplant rejection.

The antibodies to interferon gamma and/or tumor necrosis factor alpha useful in the methods of the invention may be polyclonal antibodies, monoclonal antibodies, synthetic antibodies, such as a biologically active fragment of an antibody to interferon gamma and/or tumor necrosis factor alpha, a heavy chain antibody, such as a camelid antibody, a heavy chain disease antibody, and a variable heavy chain immunoglobulin, or they may be humanized monoclonal antibodies. Methods of making and using each of the types of antibodies useful in the methods of the invention are now described. In addition, human antibodies to interferon gamma and tumor necrosis factor alpha, obtained from human donors, may be employed in the invention.

When the antibody used in the methods of the invention is a polyclonal antibody (IgG), the antibody is generated by inoculating a suitable animal with interferon gamma, tumor necrosis factor alpha, or a fragment thereof. Antibodies produced in the inoculated animal which specifically bind interferon gamma or tumor necrosis factor alpha are then isolated from fluid obtained from the animal. Interferon gamma or tumor necrosis factor alpha antibodies may be generated in this manner in several non-human mammals such as, but not limited to goat, sheep, horse, camel, rabbit, and donkey. Methods for generating polyclonal antibodies are well known in the art and are described, for example in Harlow, et al. (1988, In: Antibodies, A Laboratory Manual, Cold Spring Harbor, N.Y.). These methods are not repeated herein as they are commonly used in the art of antibody technology.

When the antibody used in the methods of the invention is a monoclonal antibody, the antibody is generated using any well known monoclonal antibody preparation procedures such as those described, for example, in Harlow et al. (supra) and in Tuszynski et al. (1988, Blood, 72:109-115). Given that these methods are well known in the art, they are not replicated herein. Generally, monoclonal antibodies directed against a desired antigen are generated from mice immunized with the antigen using standard procedures as referenced herein. Monoclonal antibodies directed against full length or peptide fragments of interferon gamma or tumor necrosis factor alpha may be prepared using the techniques described in Harlow, et al. (supra).

@@One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies.

Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The use of Old World and New World camelids for the production of antibodies is contemplated in the present invention, as are other methods for the production of camelid antibodies set forth herein.

The production of polyclonal sera from camelid species is substantively similar to the production of polyclonal sera from other animals such as sheep, donkeys, goats, horses, rabbits, mice, chickens, rats, and the like. The skilled artisan, when equipped with the present disclosure and the methods detailed herein, can prepare high-titers of antibodies from a camelid species with no undue experimentation. As an example, the production of antibodies in mammals is detailed in such references as Harlow et al., (1989, Antibodies: A Laboratory Manual, Cold Spring Harbor, N.Y.). Camelid species for the production of antibodies and sundry other uses are available from various sources, including but not limited to, Camello Fataga S. L. (Gran Canaria, Canary Islands) for Old World camelids, and High Acres Llamas (Fredricksburg, Tex.) for New World camelids.

The isolation of camelid antibodies from the serum of a camelid species can be performed by many methods well known in the art, including but not limited to ammonium sulfate precipitation, antigen affinity purification, Protein A and Protein G purification, and the like. As an example, a camelid species may be immunized to a desired antigen, for example an interferon gamma, IL-1, or tumor necrosis factor alpha peptide, or fragment thereof, using techniques well known in the art. The whole blood can them be drawn from the camelid and sera can be separated using standard techniques. The sera can then be absorbed onto a Protein G-Sepharose column (Pharmacia, Piscataway, N.J.) and washed with appropriate buffers, for example 20 mM phosphate buffer (pH, 7.0). The camelid antibody can then be eluted using a variety of techniques well known in the art, for example 0.15M NaCl, 0.58% acetic acid (pH 3.5). The efficiency of the elution and purification of the camelid antibody can be determined by various methods, including SDS-PAGE, Bradford Assays, and the like. The fraction that is not absorbed can be bound to a Protein A-Sepharose column (Pharmacia, Piscataway, N.J.) and eluted using, for example 0.15M NaCl, 0.58% acetic acid (pH 4.5). The skilled artisan will readily understand that the above methods for the isolation and purification of camelid antibodies are exemplary, and other methods for protein isolation are well known in the art and are encompassed in the present invention.

The present invention further contemplates the production of camelid antibodies expressed from nucleic acid. Such methods are well known in the art, and are detailed in, for example U.S. Pat. Nos. 5,800,988; 5,759,808; 5,840,526, and 6,015,695, which are incorporated herein by reference in their entirety. Briefly, cDNA can be synthesized from camelid spleen mRNA. Isolation of RNA can be performed using multiple methods and compositions, including TRIZOL (Gibco/BRL, La Jolla, Calif.) further, total RNA can be isolated from tissues using the guanidium isothiocyanate method detailed in, for example, Sambrook et al. (1989, Molecular Cloning, A Laboratory Manual, Cold Spring Harbor, N.Y.). Methods for purification of mRNA from total cellular or tissue RNA are well known in the art, and include, for example, oligo-T paramagnetic beads. cDNA synthesis can then be obtained from mRNA using mRNA template, an oligo dT primer and a reverse transcriptase enzyme, available commercially from a variety of sources, including Invitrogen (La Jolla, Calif.). Second strand cDNA can be obtained from mRNA using RNAse H and E. coli DNA polymerase I according to techniques well known in the art.

Identification of cDNA sequences of relevance can be performed by hybridization techniques well known by one of ordinary skill in the art, and include methods such as Southern blotting, RNA protection assays, and the like. Probes to identify variable heavy immunoglobulin chains (V_{HH}) are available commercially and are well known in the art, as detailed in, for example, Sastry et al., (1989, Proc. Nat'l. Acad. Sci. USA, 86:5728). Full-length clones can be produced from cDNA sequences using any techniques well known in the art and detailed in, for example, Sambrook et al. (1989, Molecular Cloning, A Laboratory Manual, Cold Spring Harbor, N.Y.).

The clones can be expressed in any type of expression vector known to the skilled artisan. Further, various expression systems can be used to express the V_{HH} peptides of the present invention, and include, but are not limited to eukaryotic and prokaryotic systems, including bacterial cells, mammalian cells, insect cells, yeast cells, and the like. Such methods for the expression of a protein are well known in the art and are detailed elsewhere herein.

The V_{HH} immunoglobulin proteins isolated from a camelid species or expressed from nucleic acids encoding such proteins can be used directly in the methods of the present invention, or can be further isolated and/or purified using methods disclosed elsewhere herein.

The present invention is not limited to V_{HH} proteins isolated from camelid species, but also includes V_{HH} proteins isolated from other sources such as animals with heavy chain disease (Seligmann et al., 1979, Immunological Rev. 48:145-167, incorporated herein by reference in its entirety). The present invention further comprises variable heavy chain immunoglobulins produced from mice and other mammals, as detailed in Ward et al. (1989, Nature 341:544-546, incorporated herein by reference in its entirety). Briefly, V_{H} genes were isolated from mouse splenic preparations and expressed in E. coli. The present invention encompasses the use of such heavy chain immunoglobulins in the treatment of various autoimmune disorders detailed herein.

As used herein, the term “heavy chain antibody” or “heavy chain antibodies” comprises immunoglobulin molecules derived from camelid species, either by immunization with an peptide and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

When the antibody used in the methods of the invention is a biologically active antibody fragment or a synthetic antibody corresponding to antibody to interferon gamma or tumor necrosis factor alpha, the antibody is prepared as follows: a nucleic acid encoding the desired antibody or fragment thereof is cloned into a suitable vector. The vector is transfected into cells suitable for the generation of large quantities of the antibody or fragment thereof DNA encoding the desired antibody is then expressed in the cell thereby producing the antibody. The nucleic acid encoding the desired peptide may be cloned and sequenced using technology which is available in the art, and described, for example, in Wright et al. (1992, Critical Rev. in Immunol. 12(3,4):125-168) and the references cited therein. Alternatively, quantities of the desired antibody or fragment thereof may also be synthesized using chemical synthesis technology. If the amino acid sequence of the antibody is known, the desired antibody can be chemically synthesized using methods known in the art.

The present invention also includes the use of humanized antibodies specifically reactive with IFN gamma and TNF alpha epitopes. These antibodies are capable of neutralizing human IFN gamma and human TNF alpha. The humanized antibodies of the invention have a human framework and have one or more complementarity determining regions (CDRs) from an antibody, typically a mouse antibody, specifically reactive with IFN gamma or TNF alpha. Thus, the humanized. gamma IFN and TNF alpha antibodies of the present invention are useful in the treatment of eye diseases, organ transplant rejection, and diseases of other organs which are characterized by an autoimmune reaction which includes overproduction and other pathological or detrimental results due to cytokines such as tumor necrosis factor alpha and interferon gamma.

When the antibody used in the invention is humanized, the antibody may be generated as described in Queen, et al. (U.S. Pat. No. 6,180,370), Wright et al., (supra) and in the references cited therein, Le, et al. (U.S. Pat. Nos. 6,284,471 and 6,277,9690 or in Gu et al. (1997, Thrombosis and Hematocyst 77(4):755-759). The method disclosed in Queen et al is directed in part toward designing humanized immunoglobulins that are produced by expressing recombinant DNA segments encoding the heavy and light chain complementarity determining regions (CDRs) from a donor immunoglobulin capable of binding to a desired antigen, such as human IFN gamma, attached to DNA segments encoding acceptor human framework regions. The method disclosed in Le, et al. describes compositions and the like for making chimeric anti-TNF alpha antibodies comprising murine and human fragments. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is, incorporated herein by reference).

Human constant region (CDR) DNA sequences from a variety of human cells can be isolated in accordance with well known procedures. Preferably, the human constant region DNA sequences are isolated from immortalized B-cells as described in WO 87/02671. CDRs useful in producing the antibodies of the present invention may be similarly derived from DNA encoding monoclonal antibodies capable of binding to human TNF alpha and IFN gamma. Such humanized antibodies may be generated using well known methods in any convenient mammalian source capable of producing antibodies, including, but not limited to, mice, rats, rabbits, or other vertebrates. Suitable cells for constant region and framework DNA sequences and host cells in which the antibodies are expressed and secreted, can be obtained from a number of sources such as the American Type Culture Collection, Manassas, Va.

In addition to the humanized IFN gamma and TNF alpha antibodies discussed above, other “substantially homologous” modifications to native IFN gamma and TNF alpha antibody sequences can be readily designed arid manufactured utilizing various recombinant DNA techniques well known to those skilled in the art. Moreover, a variety of different human framework regions may be used singly or in combination as a basis for humanizing antibodies directed at IFN gamma and TNF alpha. In general, modifications of genes may be readily accomplished using a variety of well-known techniques, such as site-directed mutagenesis (Gillman and Smith, Gene, 8, 81-97 (1979); Roberts et al., 1987, Nature, 328, 731-734).

Substantially homologous sequences to TNF alpha and IFN gamma antibody sequences are those which exhibit at least about 85% homology, usually at least about 90%, and preferably at least about 95% homology with a reference TNF alpha and IFN gamma immunoglobulin protein.

Alternatively, polypeptide fragments comprising only a portion of the primary antibody structure may be produced, which fragments possess one or more functions of IFN gamma or TNF alpha antibody. These polypeptide fragments may be generated by proteolytic cleavage of intact antibodies using methods well known in the art, or they may be generated by inserting stop codons at the desired locations in vectors comprising the fragment using site-directed mutagenesis. Biologically active fragments of antibodies include the polypeptide fragments described herein.

DNA encoding antibody to IFN gamma and TNF alpha are expressed in a host cell driven by a suitable promoter regulatory sequence which is operably linked to the DNA encoding the antibody. Typically, DNA encoding an antibody is cloned into a suitable expression vector such that the sequence encoding the antibody is operably linked to the promoter/regulatory sequence. Such expression vectors are typically replication competent in a host organism either as an episome or as an integral part of the host chromosomal DNA. Commonly, an expression vector will comprise DNA encoding a detectable marker protein, e.g., a gene encoding resistance to tetracycline or neomycin, to permit detection of cells transformed with the desired DNA sequences (U.S. Pat. No. 4,704,362).

Escherichia coli is an example of a prokaryotic host which is particularly useful for expression of DNA sequences encoding an antibody of the present invention. Other microbial hosts suitable for use include but are not limited to, Bacillus subtilis, and other enterobacteriaceae, such as selected member of Salmonella, Serratia, and various Pseudomonas species. It is possible to generate expression vectors suitable for the desired host cell wherein the vectors will typically comprise an expression control sequence which is compatible with the host cell. A variety of promoter/regulatory sequences are useful for expression of genes in these cells, including but not limited to the lactose promoter system, a tryptophan (trp) promoter system, a beta-lactamase promoter system, or a promoter system derived from phage lambda. The promoter will typically control expression of the antibody whose DNA sequence is operably linked thereto, the promoter is optionally linked with an operator sequence and generally comprises RNA polymerase and ribosome binding site sequences and the like for initiating and completing transcription and translation of the desired antibody.

Yeast is an example of a eukaryotic host useful for cloning DNA sequences encoding an antibody of the present invention. Saccharomyces is a preferred eukaryotic host. Promoter/regulatory sequences which drive expression of nucleic acids in eukaryotic cells include but are not limited to the 3-phosphoglycerate kinase promoter/regulatory sequence and promoter/regulatory sequences which drive expression of nucleic acid encoding other glycolytic enzymes.

In addition to microorganisms, mammalian tissue cell culture may also be used to express and produce an antibody of the present invention (Winnacker, 1987, “From Genes to Clones,” VCH Publishers, New York, N.Y). Eukaryotic cells are preferred for expression of an antibody and a number of suitable host cell lines have been developed in the art, including Chinese Hamster Ovary (CHO) cells, various COS cell lines, HeLa cells, preferably myeloma cell lines, and transformed B-cells or hybridomas. Expression vectors which express desired sequences in these cells can include expression control sequences, such as an origin of DNA replication, a promoter, an enhancer (Queen et al., 1986, Immunol. Rev., 89, 49-68), and necessary processing sequence sites, such as ribosome binding sites, RNA splice sites, polyadenylation sites, and transcriptional initiation and terminator sequences. Preferred expression control sequences are promoters derived from immunoglobulin genes, Simian Virus (SV) 40, adenovirus, cytomegalovirus, bovine papilloma virus and the like.

The vectors containing the DNA segments of interest can be transferred into the host cell by well-known methods, which vary depending on the type of cellular host. For example, calcium chloride transfection is commonly utilized for prokaryotic cells, whereas calcium phosphate treatment or electroporation may be used for other cellular hosts. (Sambrook et al., 1989, Molecular Cloning, A Laboratory Manual, Cold. Spring Harbor, N.Y.).

Once expressed, whole antibodies, dimers derived therefrom, individual light and heavy chains, or other forms of antibodies can be purified according to standard procedures known in the art. Such procedures include, but are not limited to, ammonium sulfate precipitation, the use of affinity columns, routine column chromatography, gel electrophoresis, and the like (see, generally, R. Scopes, “Protein Purification”, Springer-Verlag, N.Y. (1982)). Substantially pure antibodies of at least about 90% to 95% homogeneity are preferred, and antibodies having 98% to 99% or more homogeneity most preferred for pharmaceutical uses. Once purified, an antibody may then be used therapeutically.

An antibody of the invention may be used in a therapeutic setting in a pharmaceutical acceptable carrier either alone, or they may be used together with a chemotherapeutic agent such as a non-steroidal anti-inflammatory drug, a corticosteroid, or an immunosuppressant. The antibodies, or complexes derived therefrom, can be prepared in a pharmaceutically accepted dosage form which will vary depending on the mode of administration.

The invention thus embodies a novel composition comprising antibodies that bind with IFN gamma or TNF alpha for use in treatment of organ transplant rejection and eye disease. As stated above, the antibodies can be monoclonal antibodies, polyclonal antibodies, humanized monoclonal antibodies, a heavy chain antibody, or monoclonal chimeric antibodies, or a biologically active fragment of any type of antibody or cytokine antagonist herein recited. Generation of each type of antibody is discussed herein and applies to generation of antibodies for use in the novel methods of the invention. Generally, it is preferred that monoclonal humanized antibodies are used because they are non-immunogenic, and thus, will not elicit an immune response. However, any type of antibody may be used in the present invention.

The method of the invention is not intended to be limited to use of antibodies to IFN gamma and TNF alpha. Inhibitors to IFN gamma and TNF alpha are also useful in the method of the invention. Such inhibitors include, but are not limited to, peptides which block the function of IFN gamma or TNF alpha, IFN gamma receptor, TNF alpha receptor, antibodies to IFN gamma receptors, an antibody to a TNF alpha receptor, IFN beta, interleukin-10 (IL-10), and any combination thereof.

In one embodiment of the invention, treatment of organ transplant rejection comprises passing a fluid drawn from the patient over an immunosorbent comprising an autoimmune inhibitor, followed by returning the treated fluid to its source. This method is particularly suited for treating certain autoimmune conditions in which the autoimmune inhibitor cannot be administered to the patient. For example, the patient's fluid is exposed to an immunosorbent comprising an effective amount of target cells, CD4 cells, and/or DNA, to remove, neutralize or inhibit the autoantibodies in the patient's fluid, followed by returning the treated fluid to the patient. The immunosorbent for extracorporeal treatment may further comprise one or more antibodies (e.g., anti-alpha IFN antibodies, antibodies to alpha IFN receptor, anti-gamma IFN antibodies, antibodies to gamma IFN receptor, anti-TNF alpha antibodies, antibodies to TNF alpha receptor, antibodies to an HLA class II antigen or to its receptor, or immunoglobulin E (“IgE”)

To counter transplant rejection, antibodies to TNF alpha and IFN gamma, or in some cases anti-IFN gamma antibodies alone or anti-TNF-alpha antibodies alone, and the antigen of the transplanted cell or organ are placed in the immunosorbent column. Further, the present invention may he used in combination with immunosuppressive therapy to achieve the desired results.

For extracorporeal treatment, the pathogenic antibodies and/or immune lymphocytes can be removed or reduced by passing any of the previously described fluids over the prepared immunosorbent column comprising an autoimmune inhibitor.

When using whole blood, plasma, or plasma with leukocytes, one can use a blood cell separator (e.g., Cobe “Spectra”) to which the immunosorbent column is connected. See, e.g., U.S. Pat. No. 4,362,155, which is incorporated herein by reference. To remove pathological substances from joint or spinal fluids or the like, a special extracorporeal device with a small amount of immunosorbent is used. To neutralize antibodies to autoimmunogens, such as antibodies to target cells, including CD4 cells, the cells themselves or that portion of the cells containing the antigenic determinant(s) for the subject antibodies; must be placed directly in the immunosorbent column.

For the removal of compound(s) by extracorporeal immunosorption in accordance with the present invention, particles of sorbent material, such as amorphous silica or Sepharose, can be readily placed in a container to prepare the immunosorbent for the extracorporeal procedure. The container can be constructed of any material which can readily undergo steam, chemical, or gamma-irradiation sterilization. For instance, glass, polycarbonate; polystyrene, polymethylmethacrylate, polyolefins such as polyethylene and polypropylene, are all suitable.

Various ways of retaining or immobilizing sorbent material within a container are available. For instance, sorbent material may be placed between layers of retaining filters, or placed within a porous solid matrix. The solid matrix immobilizes the sorbent, while simultaneously permitting flow of blood or other fluids, and contact with the sorbent. As is readily apparent to one of ordinary skill in the art, a wide variety of structures arc available for providing suitable fluid/sorbent contact, structures which do not cause significant hemolysis. Prudent use of additional filters to retain the sorbent particles in their container is preferred. The pretreated, immobilized sorbent may be contacted with the fluid in a variety of ways, e.g., admixture, elution, and the like, which would be recognized in the art.

Although a columnar sorbent bed is contemplated in the present invention, beds of any other shape capable of functioning in the manner described herein may also be used. The length-to-diameter ratio of the sorbent bed should be selected so as to minimize any pressure drop along the bed, and to ensure that shear rates remain below the known values that correlate with cellular damage or destruction. The pressure drop along the sorbent bed (and thus the increase in shear rate) is directly proportional to the length of the bed. However, mitigating against use of a short bed is the fact that clearance of a substance from the fluid increases with a longer bed. The capability of the sorbent to adsorb can be assessed by experiments in which a test solution (such as whole blood or plasma) is contacted with the prepared sorbent at a constant temperature. The data generated from such an experiment can be used to determine an equilibrium constant (K), according to which the capacity of the prepared sorbent is determined. An equilibrium constant (K) is defined in units of (ml solution/g composition). The capacity of a composition provides a way to estimate the mass of the prepared sorbent required to remove a certain quantity of material, such as a cytokine, from solution.

In one embodiment of the invention, one skilled in the art will readily recognize that the disclosed autoimmune inhibitor or immunosorbent comprising the autoimmune inhibitor of the present invention can readily be incorporated into one of the established kit formats which are well known in the art. While in yet another embodiment of the present invention, kits are provided which contain the necessary reagents to carry out the previously described methods. For example, in one instance such a kit comprises a pharmaceutical composition or antibody cocktail comprising the necessary autoimmune inhibitor, with or without pharmaceutically acceptable carriers, excipients and the like, in an amount suitable for administration to a patient suffering from an autoimmune disease. In another instance, such a kit comprises the autoimmune inhibitor bound to an immunosorbent that may be used for the extracorporeal treatment of autoimmune disease in a patient. In particular, such a kit comprises an effective amount to extracorporeally remove, reduce or neutralize one or more autoimmunogens from the fluid of a patient with autoimmune disease of at least one of the following: anti-gamma IFN antibodies, antibodies to gamma IFN receptor, anti-TNF alpha antibodies, and/or antibodies to TNF alpha receptor. Another preferred kit comprises an effective amount to extracorporeally remove, reduce or neutralize one or more autoantibodies from the fluid of a patient with autoimmune disease of at least one of the following: target cells, CD4 cells, or DNA. While, yet additional kits comprise components of each of the previously defined kits, to provide the combined treatments of the present invention.

Moreover, the invention provides for the treatment of a patient with autoimmune disease by the use (administration or use in extracorporeal immunosorbent) of one or more antisense molecules, which are characterized by the ability to bind to the autoimmunogen, or a functionally equivalent derivative, or allelic or species variant thereof.

When introduced into the patient, the antisense molecule binds to, neutralizes or inhibits the autoimmunogen, much the same as an antibody. Thus, the present methods can be practiced by means of one or more antisense molecules. Moreover, when the nucleic acid sequence encoding the autoimmune anti-sense molecule is introduced into the cells under the control of a promoter, the anti-sense gene molecule binds to, neutralizes or inhibits the gene(s) encoding the autoimmunogen(s), inhibiting or preventing further pathogenesis. The inhibition appears to depend on the formation of an RNA-RNA or cDNA-RNA duplex in the nucleus or in the cytoplasm. Thus, if the antisense gene is stably introduced into a cultured cell the normal processing and/or transport is affected if a sense-antisense duplex forms in the nucleus; or if antisense RNA is introduced into the cytoplasm of the cell, the expression or translation of the autoimmunogen is inhibited. Such antisense nucleic acid sequences may further include modifications which could affect the biological activity of the antisense molecule, or its manner or rate of expression. Such modifications may also, include, e.g., mutations, insertions, deletions, or substitutions of one or more nucleotides that do not affect the function of the antisense molecule, but which may affect intracellular localization. Also, the nucleic acid sequence may determine an uninterrupted antisense RNA sequence or it may include one or more introns.

The pharmaceutical composition useful for practicing the invention may be administered to deliver a dose of between one microgram per kilogram per day and one hundred milligrams per kilogram per day.

Pharmaceutical compositions that are useful in the methods of the invention may be administered topically or systemically in ophthalmic, injectable, or other similar formulations. In addition to the antibodies to IFN gamma and/or TNF alpha, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer the gamma IFN; antibodies according to the methods of the invention.

Compounds comprising antibodies to IFN gamma and an antibody to TNF alpha that can be pharmaceutically formulated and administered to an animal for treatment of autoimmune reactions in the eye and organ transplant rejection are now described.

The invention encompasses the preparation and use of pharmaceutical compositions comprising an antibody to IFN gamma and/or an antibody to TNF alpha as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include IFN gamma receptor, TNF alpha receptor, antibodies to IFN gamma receptors, an antibody to a TNF alpha receptor, IFN beta, interleukin-10 (IL-10), and any combination thereof. The isolation of human interferon gamma receptor is well known in the art, and is described in, for example, U.S. Pat. Nos. 5,578,707; 5,221,789; and 4,897,264. Recombinant production of a human interferon gamma receptor, and antibodies that specifically bind a human interferon gamma receptor are well known in the art as well, and is described in, for example, Fountoulakis et al. (1990, J. Biol. Chem. 265: 13268-13275). Also contemplated in the present invention are chimeric interferon gamma receptors, wherein the chimeric interferon gamma receptor comprises a human interferon gamma receptor fused to another protein, such as, but not limited to a human IgG fragment, or the Fc portion of a human immunoglobulin molecule (Fountoulakis et al., 1995, J. Biol. Chem. 270: 3958-3964; Mesa et al., 1995, J. Interferon Cytokine Res. 15: 309-315). Further, the skilled artisan, when equipped with the present disclosure and the methods detailed herein, will readily be able to generate monoclonal, polyclonal and heavy chain antibodies to human interferon gamma receptor, as well as species and allelic variants thereof, biologically active fragments, and the like.

In addition to the administration of an interferon gamma receptor and antibodies that specifically bind an interferon gamma receptor, the present invention encompasses the administration of soluble TNF alpha receptors, and antibodies thereto. That is, the present invention provides methods for treating organ transplant rejection by administering soluble receptors to TNF alpha, as well as antibodies to TNF alpha receptors. A soluble TNF alpha receptor is well known in the art, and isolation from humans is described in, for example, Schall et al.(1990, Cell 61: 361-370). Further, the production of a recombinant soluble TNF alpha receptor is described in, for example, Gray et al. (1990, Proc. Nat'l. Acad. Sci. USA 87: 7380-7384). The invention further encompasses the administration of antibodies to a TNF alpha receptor. Such antibodies are well known in the art, and the skilled artisan, when armed with the present invention and the disclosure set forth herein, will readily be able to produce such antibodies. Further, the production of antibodies to a TNF alpha receptor is described in, for example, Engelmann et al. (J. Biol. Chem. 1990: 265: 14497-14504). Also included in the present invention are a chimeric TNF alpha receptor, wherein the chimeric protein comprises the 75 kDa or 55 kDa TNF-alpha receptor fused to another protein, such as a human immunoglobulin molecule, or fragments thereof Such chimeric TNF-alpha receptor fusion proteins are well known in the art, and are described in, for example, Peppel et al. (1991, J. Exp. Med. 174: 1483-1489).

The present invention also encompasses the administration of peptides and. polypeptides that specifically bind IFN gamma or TNF alpha. Such peptides and polypeptides include polypeptides comprising the epitope of the antibody or biologically active fragment thereof, or a polypeptide that is functional in conferring protection in the individual suffering from autoimmune disease, or functionally conserved fragments or amino acid variants thereof. Identification of the epitope is a matter of routine experimentation. Most typically, one would conduct systematic substitutional mutagenesis of the compound molecule while observing for reductions or elimination of cytoprotective or neutralizing activity. In ,any case, it will be appreciated that due to the size of many of the antibodies, most substitutions will have little effect on binding activity. The great majority of variants will possess at least some cytoprotective or neutralizing activity, particularly if the substitution is conservative. Conservative amino acid substitutions are substitutions from the same class, defined as acidic (Asp, Glu), hydroxy-like (Cys, Ser, Thr), amides (Asn, Gln), basic (His, Lys, Arg), aliphatic-like (Met, Ile, Leu, Val, Gly, Ala, Pro), and aromatic (Phe, Tyr, Trp).

Homologous antibody or polypeptide sequences generally will be greater than about 30 percent homologous on an identical amino acid basis, ignoring for the purposes of determining homology any insertions or deletions from the selected molecule in relation to its native sequence. The compounds discussed herein, i.e., autoimmune inhibitors for administration to the patient with autoimmune disease in accordance with the present invention, also include glycosylation variants as well as unglycosylated forms of the agents, fusions of the agents with heterologous polypeptides, and biologically active fragments of the agents, again as long as the variants possess the requisite neutralizing or cytoprotective activity.

The present invention further comprises the administration of a cytokine to inhibit the synthesis, activity, or action of interferon gamma and/or tumor necrosis factor alpha. That is, the present invention comprises a method to treat organ transplant, rejection by administering a composition that inhibits the actions, synthesis, or activity of interferon gamma and/or tumor necrosis factor alpha. The method comprises administering to a patient recombinant or otherwise purified IL-10. IL-10 is administered at a, dose of about 4 to 8 micrograms per kilogram of body weight daily for about one week to about three months. The skilled artisan, when equipped with the present invention and the methods detailed herein will readily be able to modify the dosing amount and schedule of IL-10 in order to effectively treat rejection of an organ transplant. Recombinant IL-10 is well known in the art, and is available commercially from a variety of suppliers, such as Schering-Plough (Kenilworth, N.J.).

Controlled- or sustained-release formulations of a pharmaceutical composition of the invention may be made using conventional technology.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein. Ionophoretic administration of the pharmaceutical composition of the invention is considered a form of topical administration herein.

The pharmaceutical compositions of the present invention may also be PEGylated. Procedures for coupling such moieties to a molecule are well known in the art. For example, the antibody of the present invention may be PEGylated prior to administration to a patient. Polyethylene glycol (PEG) moieties are attached to the antibody by a covalent attachment. Methods for PEGylation of antibodies are well known in the art, and are described in, for example, Choy et al. (2002, Rheumatology 41: 1133-1137). Methods for PEGylation of cytokine receptors, including IFN gamma receptor and TNF alpha receptor are similarly well known in the art, and are described in, for example, Bush et al. (2002, Scand. J. Rheumatol. 31: 198-204). General methods for PEGylating immunoglobulins, soluble receptors, and the like are described in, for example, Chapman (2002, Adv. Drug. Deliv. Review 54: 531-545).

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parenterally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e. about 20° C.) and which is liquid at the rectal temperature of the subject (i.e. about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for ophthalmic administration. Such formulations may, for example, be in the form of eye drops including, for example, a 0.1% to 1.0% (w/w) solution or suspension of the active ingredient in an aqueous or oily liquid carrier. Such drops may further comprise buffering agents, salts, or one or more other of the additional ingredients described herein. Other administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

Preferably, the composition of the invention is administered topically. The composition may be administered as an ointment to the lower eyelid. Preferably, the composition is administered in the form of eye drops. However, the composition comprising antibody to IFN-gamma and/or an antibody to TNF alpha may also be administered parenterally.

The antibodies to IFN-gamma may be present in a composition to be administered to the affected eye at a range of concentrations.

A composition comprising an antibody to IFN gamma can be administered to the affected eye several times per day. Preferably, the composition is administered from one to five times per day, and more preferably, the composition is administered from one to three times per day. Most preferred is administration of the composition three times per day.

IFN gamma antibodies can be administered to the affected eye of a patient for as long as necessary to remedy the effects of the autoimmune reaction. Preferably, the patient receives treatment for about 5 to about 10 days. More preferably, the patient receives treatment for about 5 to about 7 days. The entire treatment of administering IFN gamma antibodies can be repeated.

As evidenced by the Examples, the present invention is particularly useful in treating a hyperimmune response resulting from rejection of an eye-related tissue or organ transplant. The invention is also useful in preventing an expected rejection of a transplanted tissue or organ when the composition of the invention is administered about one day before, during, and immediately after transplant surgery. The preferred treatment period is about seven days.

Administering IFN gamma antibodies to the an affected eye is also effective against damage of eye and optic nerve cells caused by hyperproduction of IFN gamma. Hyperproduction of IFN gamma can also induce an autoimmune response in the eye. Thus, the administration of IFN gamma antibodies to an eye affected with a disease that causes hyperproduction of IFN gamma is well within the purview of the present invention.

The present invention further encompasses methods for treating organ transplant rejection. Organ transplant rejection includes, but is not limited to, hyperacute rejection, acute rejection, acute vascular rejection, acute cellular rejection, and chronic rejection. This is because, as disclosed herein, cytokine expression and secretion in a patient experiencing organ transplant rejection are related to effector cell activation, fibrosis, anti-organ antibodies, and other mechanisms of organ transplant rejection. The present invention comprises a method to treat organ transplant rejection in a human patient, the method comprising administering to a patient an antibody that specifically binds TNF alpha and an antibody that specifically binds IFN gamma, either alone, or in combination. Administration of an antibody to TNF alpha and/or an antibody to IFN gamma results in the alleviation of organ transplant rejection, including, but not limited to, reduced or lost organ function, pain or swelling at the location of the organ, fever, malaise, and the like. Further, given the shortage of organs available for transplantation, the methods of the present invention provide for a lower chance, if any, of rejection, and a longer survival time of both the patient and the organ, thereby decreasing the need for multiple transplants in the same patient.

The method comprises administering an antibody to TNF alpha and an antibody to IFN gamma, alone or in combination with each other, to a patient whose body is rejecting an organ transplant. The antibody is administered in an effective amount, as disclosed elsewhere herein. As an example, antibodies to TNF alpha and/or IFN gamma can be administered intramuscularly, intravenously, intradermally, cutaneously, subcutaneously, ionophoretically, topically, locally, and by inhalation, preferably by parenteral administration. The concentration of anti-TNF-alpha antibodies can be from about 1 μg/ml to about 500 μg/ml, preferably from about 10 μg/ml to about. 200 μg/ml, even more preferably from about 20 μg/ml to about 100 μg/ml, yet more preferably from about 30 μg/ml to about 75 μg/ml, preferably about 66 μg/ml. The amount of anti-TNF alpha antibody and/or anti-IFN gamma antibody administered to a patient can be from about 0.1 ml to about 10 ml, preferably from about 0.5 ml to about 7 ml, more preferably from about 1 ml to about 5 ml, even more preferably about 2 ml. Preferably, a patient is administered an anti-TNF alpha antibody dose of about 1 mg/kg of body weight to about 10 mg/kg of body weight for the first 1 to about 5 days of treatment. Preferably a patient is administered an anti-IFN gamma antibody dose of from about 5 to about 15 mg/kg of body weight for the first 1 to about 5 days of treatment. This dose of doses can be repeated, depending on the results of the treatment detailed herein and well known to the skilled artisan equipped with the present disclosure.

The present invention further comprises the administration of antibodies to IFN gamma receptors and antibodies to TNF-alpha receptors for the treatment of organ transplant rejection in a human. Antibodies to cytokine receptors, in particular a TNF alpha receptor and/or an IFN gamma receptor, are administered using the routes and methods disclosed elsewhere herein. An anti-TNF alpha receptor antibody and/or an anti-IFN gamma receptor antibody are administered at a dosage of about 0.2 to about 15 mg/kg of body weight every week for about 2 to about 6 weeks. The dosage of anti-cytokine receptor antibody can be altered according to the knowledge of the skilled artisan using the guidelines set forth elsewhere herein.

The invention also encompasses the administration of soluble cytokine receptors for the treatment of organ transplant rejection in a human. Soluble cytokine receptors, specifically soluble cytokine receptors that bind to IFN gamma and/or TNF alpha are administered using the methods detailed herein and well known in the art. As a non-limiting example, a soluble receptor that binds TNF alpha or a soluble receptor that binds IFN gamma, administered alone or in combination, is administered at a dose of about 25 mg/kg of body weight for about a week. This regimen is continued for about 24 weeks, and can be altered depending on the age of the patient and the clinical circumstances well known in the art and detailed elsewhere herein.

The concentration and dosage of an antibody can be altered depending on age and clinical situation. As is well known in the art; for children under about 15 years old and for adults over about 75 years old, the concentration of antibody is reduced by about half.

An anti-TNF alpha and/or anti-IFN gamma antibody can be administered from about once a year to about twice per year to several times a year to monthly to a few times a month to several times a month to weekly to several times a week to daily, to twice daily to several times a day. Preferably, the anti-TNF alpha antibody and the anti-IFN gamma antibody, administered alone or in combination, is administered to a patient about twice daily for about five consecutive days. This process may be repeated, as can be determined by one of skill in the art. Criteria for repeating the process, increasing the dose of anti-cytokine antibody, and the like are well known in the art and include monitoring the organ function, monitoring pain, swelling or discomfort at the location of the transplanted organ, monitoring the patient's temperature, and observing signs of general discomfort, uneasiness, an ill feeling, or malaise in a patient. Further, signs of organ rejection and the success of the methods detailed herein can be monitored using standard histopathology methods well known in the art, or immune effector mechanism assays well known in the art, such as serum cytokine levels, anti-organ antibody levels, and anti-organ lymphocyte production. Methods for measuring the level of transplanted organ are well known in the art, and include, but are not limited to, laboratory tests for renal function and liver function, ultrasound of the transplanted organ, renal arteriography, production of insulin, c-peptide levels, abdominal CT scans, cardiac echogram, chest X-ray, and other methods of organ function well known in the art.

Methods for recognizing and diagnosing organ transplant rejection are well known in the art and are described in, for example, Solid Organ Transplant Rejection, Solez. et al. (Eds.) (1996, Marcel Dekker, New York, N.Y.).

As discussed elsewhere herein, an antibody to TNF alpha and/or IFN gamma comprises a polyclonal antibody, a monoclonal antibody, a humanized antibody, a camelid or heavy chain antibody, and a synthetic antibody. The present invention further encompasses a biologically active fragment of an antibody, a functional equivalent of an antibody, a derivative of an antibody, an allelic variant of an antibody, and a species variant of an antibody. The antibodies, fragments, equivalents, derivatives, and variants thereof necessary to practice the methods of the present invention will be apparent to one of skill in the art when supplied with the present disclosure. The skilled artisan will further appreciate that the present invention is not limited to the singular administration of an antibody, fragment, equivalent, derivative, or variant thereof, but rather that they may be administered in a combination, either in combination with each other or in a temporal sense. As an example, solid organ transplant rejection can be treated with an, antibody to TNF alpha, an antibody to IFN gamma, or a combination of antibodies comprising an antibody to IFN gamma and an antibody to TNF alpha.

The method of the present invention further includes routes in which to administer an antibody to TNF alpha and/or an antibody to IFN gamma to a patient. The skilled clinician will recognize that routes of administration may vary, depending on the status and needs of the patient, the resources available, the severity of the disease, and the like. However, as amply disclosed by the teachings provided herein, the route of administration can include, but is not limited to intramuscular, intravenous, intradermal, cutaneous, subcutaneous, ionophoretical, topical, local, and inhalation administration. Thereby, the skilled artisan will be able to easily determine the best route of administration for the patient experiencing solid organ transplant rejection based on the patient's status, the severity of the organ transplant rejection, and other indicators well known to the skilled artisan.

As a non-limiting example, a patient diagnosed with organ transplant rejection, according to the methods disclosed herein, can be treated as follows. The patient is administered a series of tests to determine organ function using methods disclosed elsewhere herein. Further, the patient is evaluated for other signs of solid organ transplant rejection, including, but not limited to pain or swelling at the transplanted organ location, the presence of fever, and a feeling of discomfort or malaise. In addition, baseline levels of the circulating cytokines, such as TNF alpha and IFN gamma, can be assessed using standard clinical laboratory tests, including ELISA and other immunoblot tests well known in the art.

The patient is then administered anti-TNF alpha antibodies, anti-IFN gamma antibodies, or both anti-TNF alpha antibodies and anti-IFN gamma antibodies. Preferably, the activity of the antibodies is measured prior to administration to,the patient, and the levels are within limits well known in the art and described elsewhere herein. The anti-TNF alpha and/or anti-IFN gamma antibodies are administered parenterally, preferably intramuscularly or intravenously to a patient. Administration takes place over a series of days, preferably two injections of antibody per day for five consecutive days. This process may be repeated based on clinical results and the patient's ability to tolerate the treatment. The process can also include the administration of an immunosuppressive agent described elsewhere herein or well known in the art. Such immunosuppressive agents include, but are not limited to corticosteroids such as prednisolone, and other agents such as cyclosporine, tacrolimus, azathioprine, cyclophosphamide, and the like. However, as will be readily recognized by one of skill in the art, the doses and frequency of administration of these conventional immunosuppressive agents can be reduced when administered in conjunction with the antibodies of the present invention thus reducing the chance of chronic immunosuppression-mediated infection and the toxicity of conventional immunosuppressive agents detailed elsewhere herein;. As a non-limiting example, a patient is administered an antibody to IFN gamma or an antibody to TNF alpha in combination with a corticosteroid such as prednisolone, or another generalized immunosuppressant such as cyclosporine, tacrolimus, azathioprine, cyclophosphamide, or others known in the art and described herein. The concentration and dose of an antibody administered to a patient can be reduced when co-administered with an immunosuppressant. That is, the dose of the antibody and the dose of the immunosuppressant can be altered when an antibody an immunosuppressant are administered to a patient suffering from organ transplant rejection. The doses and/or concentration of the antibody and immunosuppressant can be altered proportionally to each other. As an example, the dose of the antibody, as described elsewhere herein, can be reduced by half, and the dose of the immunosuppressant, as disclosed herein and as known in the art, can be reduced by half as well. However, as will be readily apparent to the skilled artisan, the dose of an antibody and an immunosuppressant can be altered in a non-proportional manner, according to the patient's status and efficacy of the therapy.

Organ function tests are administered at intervals following antibody administration. These results of these tests are compared to baseline readings of organ function to evaluate progress. Further, the patient's overall well-being is monitored through other psychosocial parameters, such as pain, swelling or discomfort at the site of transplantation, fever, malaise, and the like. The patient's medical condition is monitored for the appearance of rashes or allergic reactions to anti-TNF alpha, anti-IFN gamma or combination therapy. Such reactions may indicate that the treatment should be postponed, or if mild, the treatment can be continued along with therapies to alleviate rashes and allergic reactions, such as low-dose topical steroids, antihistamines, and the like. Recognition and management of rashes and other reactions are well within the abilities of one of ordinary skill in the art. Organ function and other medical indicators of treatment of'solid organ transplant rejection and determination of the circulating level of TNF alpha and/or IFN gamma are monitored throughout the patient's treatment to determine the progress of the therapy. Further, continuous monitoring allows the clinician to determine if therapy is effective and if administration should continue.

The present invention also includes methods for treating organ transplant rejection with a combination therapy. Such solid organ transplant rejection includes, but is not limited to, hyperacute rejection, acute rejection, acute vascular rejection, acute cellular rejection, chronic rejection, and the like. This is because, as disclosed herein, administration of an effective amount of an antibody to IFN gamma and TNF alpha is useful in treating organ transplant rejection in a human patient.

The method comprises administering antibodies to IFN gamma and TNF alpha to a patient undergoing organ transplant rejection. The antibodies are administered in an effective amount, which will be readily apparent of one of skill in the art when equipped with the present disclosure and the teachings herein. Further, the skilled clinician will be able to recognize organ transplant rejection when armed with the present disclosure.

As discussed elsewhere herein, an antibody comprises a polyclonal antibody, a monoclonal antibody, a humanized antibody, a camelid antibody or heavy chain antibody, and a synthetic antibody. The present invention further encompasses a biologically active fragment of an antibody, a functional equivalent of an antibody, a derivative of an antibody, an allelic variant of an antibody, and a species variant of an antibody. The antibodies, fragments, equivalents, derivatives, and variants thereof necessary to practice the methods of the present invention will be apparent to one of skill in the art when supplied with the present disclosure. The skilled artisan will further appreciate that the present invention is not limited to the singular administration of an antibody, fragment, equivalent, derivative, or variant thereof, but rather that they may be administered in a combination, either in combination with each other or in a temporal sense. The method of the present invention further includes routes in which to administer antibodies to IFN gamma and TNF alpha to a patient. The skilled clinician will recognize that routes of administration may vary, depending on the status and needs of the patient, the resources available, the severity of the transplant rejection (e.g. hyperacute, acute, or chronic), and the like. However, as amply disclosed by the teachings provided herein, the route of administration can include, but is not limited to intramuscular, intravenous, intradermal, cutaneous, subcutaneous, ionophoretical, topical, local, and inhalation administration. Thereby, the skilled artisan will be able to easily determine the best route of administration with little or no undue experimentation.

The invention is now described with reference to the following Examples. These Examples are provided for the purpose of illustration only and the invention should, in no way be construed as being limited to these Examples, but rather should be construed to encompass any and all variations which become evident as a result of the teaching provided herein.

Definitions

The articles “a” and “an” are used herein to refer to one or to more than one (i.e. to at least one) of the grammatical object of the article. By way of example, “an element” means one element or more than one element.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated b the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

“Antisense sequence,” or “antisense molecule” refers to peptides derived from pseudogenes which are constructed by reversing the orientation of the gene encoding the autoimmunogen with regard to its promoter, so that the antisense strand is transcribed. The term also refers to the antisense strand of RNA or of cDNA which compliments the strand of DNA encoding the cytokine, autoimmunogen, protein or peptide of interest.

By the term “biologically active antibody fragment” is meant a fragment of an antibody which retains the ability to specifically bind to a cytokine, such as IFN gamma, TNF alpha, and IFN alpha. Biologically active fragments include, but are not limited to Fv, Fab and F(ab)₂ fragments of antibodies, as well as other fragments of antibodies that retain the ability to bind a cytokine.

“Camelid” is used herein to refer to members of the order Artiodactyla including Old World camels such as the one-humped Arabian Camel, Camelus dromedarius and the twin-humped Bactrian camel C. bactrianus. Camelids, as used herein also refers to New World camels, including llamas, alpacas, guanacos, and vicunas.

A “camelid antibody” is used herein to refer to an immunoglobulin molecule naturally present in a camelid species, or a derivative of an immunoglobulin molecule naturally present in a camelid species where the derivative retains some portion of the amino acid sequence present in a naturally occurring immunoglobulin present in a camelid species.

A “disease” is a state of health of an animal wherein the animal cannot maintain homeostasis, and wherein if the disease is not ameliorated then the animal's health continues to deteriorate. Use of the term disease throughout the application is meant to encompass the terms diseases, disorders, and conditions.

A “heavy chain disease antibody” as used herein refers to an immunoglobulin molecule derived from a mammal with a disorder in which the amino acid sequences harbors a deletion of one or more amino acids in the variable domain through the first domain of the constant region such that cross-links to the light chain of the antibody are not formed. Such as disorder is known as heavy chain disease.

“Immunosuppressant” is used herein to refer to a compound that is administered to detectably lower the level of immune system reaction to a foreign or auto-antigen. Immunosuppressants include, but are not limited to, cyclosporine, tacrolimus, azathioprine, cyclophosphamide, and prednisolone, prednisone, and other corticosteroids.

“Organ” or “solid organ” is used herein to refer to a part of the body having a function in the homeostasis of the body, wherein the part of the body is not skin.

“Transplant rejection” is used herein to refer to the process of the organ recipient's immune system attacking, or otherwise inhibiting the acceptance of the donated organ into the recipient's body, otherwise known as host versus graft disease.

“Treatment” of a disease occurs when the severity of a symptom of the disease, the frequency with which such a symptom is experienced by a patient, or both, is reduced or eliminated. “Treatment” also encompasses prevention of an anticipated disease state. For example, treatment of a transplant rejection includes use of a composition comprising antibodies to IFN gamma after rejection has already occurred, and also within a period of post-transplant surgery to prevent an anticipated rejection. The preferred period post-surgery is about seven days.

By the term “specifically binds,” as used herein, it is meant an antibody which recognizes and binds a specific cytokine, such as IFN gamma, IFN alpha, and TNF alpha, but does not substantially recognize or bind other molecules or cytokines in a sample.

“Variable heavy chain immunoglobulin” used herein to refer to an immunoglobulin molecule prepared from the variable region of the heavy chain of an animal immunized with an antigen. Such immunoglobulin molecules retain the ability to bind to the immunizing antigen.

“Autoimmune response” refers to an alteration in the immune system wherein the immune response mounted during a disease state is detrimental to the host. Typically, cells of the immune system or other immune system components such as antibodies produced by the host, recognize “self” antigens as foreign antigens.

A “hyperimmune response” refers to an autoimmune response characterized by an overexpression of one or more cytokines of the immune system.

As used here, “an eye-related tissue or organ” refers to the tissues and organs that constitute the eye. These include all parts of the eye as would be classified in an anatomy textbook, for example, Williams et al., eds., 1980, Gray's Anatomy, 36th ed., W. B. Saunders Co., Philadelphia.

A “corneal transplant” refers to the insertion of a cornea into the eye of a mammal, where the cornea being inserted is not the natural cornea of the mammal. The cornea being inserted may be from a cadaver.

A pharmaceutical composition is said to be “topically administered” when it is applied directly to the affected area. Eye drops, for example, are applied topically, as are creams and ointments. Ionophoresis is also included as a form of topical administration.

“Recombinant DNA” refers to a polynucleotide having sequences that are not naturally joined together. An amplified or assembled recombinant polynucleotide may be included in a suitable vector, and the vector can be used to transform a suitable host cell. A recombinant DNA polynucleotide may serve a non-coding function (e.g., promoter, origin of replication, ribosome-binding site, etc.) as well.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

In each of the trials reported below, the concentration of Fab2 fragments of antibody was 50 mg/ml of protein. The anti-IFN gamma activity when measured by ELISA exhibited a significant signal at a dilution of 1:10,000. The fragments were in liquid form. The liquid formulation of antibody fragments was administered at two to three drops per eye, three times per day for seven to ten days. Improvements in visual acuity and other signs were noted often by the second or third day after administration of the drops.

Clinical trials were conducted with on three patients who had recently, undergone corneal transplant surgery. Patient G, male, fifty-three years of age, underwent corneal transplantation to treat keratoconus. The surgery included extraction of a cataract and implantation of an artificial lens. Patient G subsequently had a transplant rejection reaction characterized by deteriorating vision and opacity of the corneal transplant. Patient G was treated with standard therapy without therapeutic effect. Standard therapies may be steroids, anti-inflammatories, antibiotics, or vitamins, or any combination thereof, administered either topically, in the form of drops or ointment, or intravenously, by injection under the conjunctiva, orally, and intramuscularly. Fragments of goat anti-human interferon gamma antibodies were administered to the affected eye, in the form of eye drops on an outpatient basis. The drops were administered at two drops three times daily, over a period of seven days. Patient G exhibited a significant improvement in visual acuity after two days of treatment. Further, the corneal transplant reverted from opacity to almost complete transparency and peripheral areas of the cornea became significantly more transparent as well.

Patient P, male, thirty-nine years of age, underwent corneal transplantation to treat keratoconus in 1999. Nine months later, Patient P was diagnosed with a transplant rejection reaction and was treated with twenty-five doses of dexamethasone, both intravenously and using,eye drops. Patient P received other types of therapy as well, and continued treatment on an outpatient basis. Six months after the first transplant rejection, Patient P was diagnosed with a second transplant rejection reaction. Patient P was treated on an outpatient basis with the same therapy used for the first rejection. One month later, Patient P's previous therapy was discontinued and treatment with antibodies to interferon gamma in the form of eye drops was initiated. One day later, Patient P experienced improvement in visual acuity and the transplanted cornea became more transparent in peripheral areas. Over the next two days of treatment, Patient P exhibited complete corneal transparency and a drastic improvement of vision.

Patient F, female, fifty-three years of age, underwent corneal transplantation and extraction of a cataract to treat a purulent corneal ulcer and herpes zoster. Ten days later, the transplant was rejected. Patient F underwent another corneal transplantation thirteen days after rejection of the first transplant. Patient F received therapy with multiple antibiotics, steroids, anti-inflammatory preparations, and atropine. Despite all therapies administered, Patient F persistently displayed a purulent ring around the transplant, the transplant itself was cloudy, and the anterior eye chamber was hemorrhaging and was filled with exudate. Patient F's affected eye was treated with antibodies to interferon gamma in the form of eye drops, administered at 2 drops three times daily. After three days of administration, Patient F's condition improved. The purulent ring around the transplant significantly cleared and became white and the cornea became significantly more transparent. Exudate and hemorrhage in the anterior chamber completely disappeared, and the affected eye appeared significantly normal.

The results of the experiments disclosed establish that treatment of hyperimmune disease of the eye with antibody to IFN gamma is effective.

The disclosures of each and every patent, patent application, and publication cited herein, are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations. CLAIMS

What is claimed is:

1. A method of treating organ transplant rejection in a human patient, wherein the organ is not skin, the method comprising administering to the patient an effective amount of a combination of an antibody that specifically binds interferon gamma and an antibody that specifically binds tumor necrosis factor alpha.

2. The method of claim 1, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

3. The method of claim 1, wherein the antibody is administered by the route selected from the group consisting of intramuscularly, intravenously, intradermally, cutaneously, subcutaneously, rectally, ionophoretically, topically, locally, and inhalation.

4. The method of claim 1, wherein the antibody is selected from the group consisting of a polyclonal antibody, a monoclonal antibody, a synthetic antibody, a heavy chain antibody and a humanized antibody.

5. The heavy chain antibody of claim 4, wherein the heavy chain antibody is selected from the group consisting of a camelid antibody, a heavy chain disease antibody, and a variable heavy chain immunoglobulin.

6. The method of claim 1, wherein the human patient is, optionally administered an immunosuppressant.

7. A method of treating heart transplant rejection in a human patient, the method comprising administering to the patient an effective amount of a combination of an antibody that specifically binds interferon gamma and an antibody that specifically binds tumor necrosis factor alpha.

8. The method of claim 7, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof; an allelic variant thereof, a species variant thereof, a heavy chain antibody, and combinations thereof.

9. The method of claim 7, wherein the antibody is administered by the route selected from the group consisting of intramuscularly, intravenously, intradermally, cutaneously, subcutaneously, rectally, ionophoretically, topically, locally, and inhalation.

10. The method of claim 7, wherein the antibody is selected from the group consisting of a polyclonal antibody, a monoclonal antibody, a synthetic antibody, a heavy chain antibody and a humanized antibody.

11. The heavy chain antibody of claim 10, wherein the heavy chain antibody is selected from the group consisting of a camelid antibody, a heavy chain disease antibody, and a variable heavy chain immunoglobulin.

12. The method of claim 7, wherein the human patient is optionally administered an immunosuppressant.

13. A method of treating kidney transplant rejection in a human patient, the method comprising administering to the patient an effective amount of a combination of an antibody that specifically binds interferon gamma and an antibody that specifically binds tumor necrosis factor alpha.

14. The method of claim 13, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

15. The method of claim 13, wherein the antibody is administered by the route selected from the group consisting of intramuscularly, intravenously, intradermally, cutaneously, subcutaneously, rectally, ionophoretically, topically, locally, and inhalation.

16. The method of claim 13, wherein the antibody is selected from the group consisting of a polyclonal antibody, a monoclonal antibody, a synthetic antibody, a heavy chain antibody and a humanized antibody.

17. The heavy chain antibody of claim 16, wherein the heavy chain antibody is selected from the group consisting of a camelid antibody, a heavy chain disease antibody, and a variable heavy chain immunoglobulin.

18. The method of claim 13, wherein the human patient is optionally administered an immunosuppressant.

19. A method of treating liver transplant rejection in a human patient, the method comprising administering to the patient an effective amount of a combination of an antibody that specifically binds interferon gamma and an antibody that specifically binds tumor necrosis factor alpha.

20. The method of claim 19, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

21. The method of claim 19, wherein the antibody is administered by the route selected from the group consisting of intramuscularly, intravenously, intradermally, cutaneously, subcutaneously, rectally, ionophoretically, topically, locally, and inhalation.

22. The method of claim 19, Wherein the antibody is selected from the group consisting of a polyclonal antibody, a monoclonal antibody, a synthetic antibody, a heavy chain antibody and a humanized antibody.

23. The heavy chain antibody of claim 22, wherein the heavy chain antibody is selected from the group consisting of a camelid antibody, a heavy chain disease antibody, and a variable heavy chain immunoglobulin.

24. The method of claim 19, wherein the human patient is optionally administered an immunosuppressant.

25. A method of treating pancreatic beta-islet cell transplant rejection in a human patient, the method comprising administering to the patient an effective amount of a combination of an antibody that specifically binds interferon gamma and an antibody that specifically binds tumor necrosis factor alpha.

26. The method of claim 25, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

27. The method of claim 25, wherein the antibody is administered by the route selected from the group consisting of intramuscularly, intravenously, intradermally,cutaneously, subcutaneously, rectally, ionophoretically, topically, locally, and inhalation.

28. The method of claim 25, wherein the antibody is selected from the group consisting of a polyclonal antibody, a monoclonal antibody; a synthetic antibody, a heavy chain antibody and a humanized antibody.

29. The heavy chain antibody of claim 28, wherein the heavy, chain antibody is selected from the group consisting of a camelid antibody, a heavy chain disease antibody, and a variable heavy chain immunoglobulin.

30. The method of claim 25, wherein the human patient is optionally administered an immunosuppressant.

31. A method of treating organ transplant rejection in a human patient, wherein the organ is not skin, the method comprising administering to the patient an effective amount of an antibody that specifically binds interferon gamma.

32. The method of claim 31, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

33. The method of claim 31, wherein the antibody is administered by the route selected from the group consisting of intramuscularly, intravenously, intradermally, cutaneously, subcutaneously, rectally, ionophoretically, topically, locally, and inhalation.

34. The method of claim 31, wherein the antibody is selected from the group consisting of a polyclonal antibody, a monoclonal antibody, a synthetic antibody, a heavy chain antibody and a humanized antibody.

35. The heavy chain antibody of claim 34, wherein the heavy chain antibody is selected from the group consisting of a camelid antibody, a heavy chain disease antibody, and a variable heavy chain immunoglobulin.

36. The method of claim 31, wherein the human patient is optionally administered an immunosuppressant.

37. A method of treating heart transplant rejection in a human patient, the method comprising administering to the patient an effective amount of an antibody that specifically binds interferon gamma.

38. The method of claim 37, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

39. The method of claim 37, wherein the antibody is administered by the route selected from the group consisting of intramuscularly, intravenously, intradermally, cutaneously, subcutaneously, rectally, ionophoretically, topically, locally, and inhalation.

40. The method of claim 37, wherein the antibody is selected from the group consisting of a polyclonal antibody, a monoclonal antibody, a synthetic antibody, a heavy chain antibody and a humanized antibody.

41. The heavy chain antibody of claim 40, wherein the heavy chain antibody is selected from the group consisting of a camelid antibody, a heavy chain disease antibody, and, a variable heavy chain immunoglobulin.

42. The method of claim 37, wherein the human patient is optionally administered an immunosuppressant.

43. A method of treating kidney transplant rejection in a human patient, the method comprising administering to the patient an effective amount of an antibody that specifically binds interferon gamma.

44. The method of claim 43, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

45. The method of claim 43, wherein the antibody is administered by the route selected from the group consisting of intramuscularly, intravenously, intradermally, cutaneously, subcutaneously, rectally, ionophoretically, topically, locally, and inhalation.

46. The method of claim 43, wherein the antibody is selected from the group consisting of a polyclonal antibody, a monoclonal antibody, a synthetic antibody, a heavy chain antibody and a humanized antibody.

47. The heavy chain antibody of claim 46, wherein the heavy chain antibody is selected from the group consisting of a camelid antibody, a heavy chain disease antibody, and a variable heavy chain immunoglobulin.

48. The method of claim 43, wherein the human patient is optionally administered an immunosuppressant.

49. A method of treating liver transplant rejection in a human patient, the method comprising administering to the patient an effective amount of an antibody that specifically binds interferon gamma.

50. The method of claim 49, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

51. The method of claim 49, wherein the antibody is administered by the route selected from the group consisting of intramuscularly, intravenously, intradermally, cutaneously subcutaneously, rectally, ionophoretically, topically, locally, and inhalation.

52. The method of claim 49, wherein the antibody is selected from the group consisting of a polyclonal antibody, a monoclonal antibody, a synthetic antibody, a heavy chain antibody and a humanized antibody.

53. The heavy chain antibody of claim 52, wherein the heavy chain antibody is selected from the group consisting of a camelid antibody, a heavy chain disease antibody, and a variable heavy chain immunoglobulin.

54. The method of claim 49, wherein the human patient is optionally administered an immunosuppressant.

55. A method of treating pancreatic beta-islet cell transplant rejection in a human patient, the method comprising administering to the patient an effective amount of an antibody that specifically binds interferon gamma.

56. The method of claim 55, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

57. The method of claim 55, wherein the antibody is administered by the route selected from the group consisting of intramuscularly, intravenously, intradermally, cutaneously, subcutaneously, rectally, ionophoretically, topically, locally, and inhalation.

58. The method of claim 55, wherein the antibody is selected from the group consisting of a polyclonal antibody, a monoclonal antibody, a synthetic antibody, a heavy chain antibody and a humanized antibody.

59. The heavy chain antibody of claim 58, wherein the heavy chain antibody is selected from the group consisting of a camelid antibody, a heavy chain disease antibody, and a variable heavy chain immunoglobulin.

60. The method of claim 55, wherein the human patient is optionally administered an immunosuppressant.

61. A method of treating organ transplant rejection in a human patient, wherein the organ is not skin, the method comprising administering to the patient an effective amount of an antibody that specifically binds tumor necrosis factor alpha.

62. The method of claim 61, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

63. The method of claim 61, wherein the antibody is administered by the route selected from the group consisting of intramuscularly, intravenously, intradermally, cutaneously, subcutaneously, rectally; ionophoretically, topically, locally, and inhalation.

64. The method of claim 61, wherein the antibody is selected from the group consisting of a polyclonal antibody, a monoclonal antibody, a synthetic antibody, a heavy chain antibody and a humanized antibody.

65. The heavy chain antibody of claim 64, wherein the heavy chain antibody is selected from the group consisting of a camelid antibody, a heavy chain disease antibody, and a variable heavy chain immunoglobulin.

66. The method of claim 61, wherein the human patient is optionally administered an immunosuppressant.

67. A method of treating heart transplant rejection in a human patient, the method comprising administering to the patient an effective amount of an antibody that specifically binds tumor necrosis factor alpha.

68. The method of claim 67, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

69. The method of claim 67, wherein the antibody is administered by the route selected from the group consisting of intramuscularly, intravenously, intradermally, cutaneously, subcutaneously, rectally, ionophoretically, topically, locally, and inhalation.

70. The method of claim 67, wherein the antibody is selected from the group consisting of a polyclonal antibody, a monoclonal antibody, a synthetic antibody, a heavy chain antibody and a humanized antibody.

71. The heavy chain antibody of claim 70, wherein the heavy chain antibody is selected from the group consisting of a camelid antibody, a heavy chain disease antibody, and a variable heavy chain immunoglobulin.

72. The method of claim 67, wherein the human patient is optionally administered an immunosuppressant.

73. A method of treating kidney transplant rejection in a human patient, the method comprising administering to the patient an effective amount of an antibody that specifically binds tumor necrosis factor alpha.

74. The method of claim 73, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

75. The method of claim 73, wherein the antibody is administered by the route selected from the group consisting of intramuscularly, intravenously, intradermally, cutaneously, subcutaneously, rectally, ionophoretically, topically, locally, and inhalation.

76. The method of claim 73, wherein the antibody is selected from the group consisting of a polyclonal antibody, a monoclonal antibody, a synthetic antibody, a heavy chain antibody and a humanized antibody.

77. The heavy chain antibody of claim 76, wherein the heavy chain antibody is selected from the group consisting of a camelid antibody, a heavy chain disease antibody, and a variable heavy chain immunoglobulin.

78. The method of claim 73 wherein the human patient is optionally administered an immunosuppressant.

79. A method of treating liver transplant rejection in a human patient, the method comprising administering to the patient an effective amount of an antibody that specifically binds tumor necrosis factor alpha.

80. The method of claim 79, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

81. The method of claim 79, wherein the antibody is administered by the route selected from the group consisting of intramuscularly, intravenously, intradermally, cutaneously, subcutaneously, rectally, ionophoretically, topically, locally, and inhalation.

82. The method of claim 79, wherein the antibody is selected from the group consisting of a polyclonal antibody, a monoclonal antibody, a synthetic antibody, a heavy chain antibody and a humanized antibody.

83. The heavy chain antibody of claim 82, wherein the heavy chain antibody is selected from the group consisting of a camelid antibody, a heavy chain disease antibody, and a variable heavy chain immunoglobulin.

84. The method of claim 79, wherein the human patient is optionally administered an immunosuppressant.

85. A method of treating pancreatic beta-islet cell transplant rejection in a human patient, the method comprising administering to the patient an effective amount of an antibody that specifically binds tumor necrosis factor alpha.

86. The method of claim 85, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

87. The method of claim 85, wherein the antibody is administered by the route selected from the group consisting of intramuscularly, intravenously, intradermally, cutaneously, subcutaneously, rectally, ionophoretically, topically, locally, and inhalation.

88. The method of claim 85, wherein the antibody is selected from the group consisting of a polyclonal antibody, a monoclonal antibody, a synthetic antibody, a heavy chain antibody and a humanized antibody.

89. The heavy chain antibody of claim 88, wherein the heavy chain antibody is selected from the group consisting of a camelid antibody, a heavy chain disease antibody, and a variable heavy chain immunoglobulin.

90. The method of claim 85, wherein the human patient is optionally administered an immunosuppressant.

91. A method of treating lung transplant rejection in a human patient, the method comprising administering to the patient an effective amount of a combination of an antibody that specifically binds tumor necrosis factor alpha and an antibody that specifically binds interferon gamma.

92. The method of claim 91, wherein the antibody is selected from the group consisting of a polyclonal antibody, a biologically active fragment thereof, an allelic variant thereof, a species variant thereof, a monoclonal antibody, a biologically active fragment thereof, an allelic variant thereof a species variant thereof, a humanized antibody, a biologically active fragment thereof, an allelic variant thereof a species variant thereof, a synthetic antibody, a biologically active fragment thereof, an allelic variant thereof, a species variant thereof a heavy chain antibody, and combinations thereof.

93. The method of claim 91, wherein the antibody is administered by the route selected from the group consisting of intramuscularly, intravenously, intradermally, cutaneously, subcutaneously, rectally, ionophoretically, topically, locally, and inhalation.

94. The method of claim 91, wherein the antibody is selected from the group consisting of a polyclonal antibody, a monoclonal antibody, a synthetic antibody, a heavy chain antibody and a humanized antibody.

95. The heavy chain antibody of claim 94, wherein the heavy chain antibody is selected from the group consisting of a camelid antibody, a heavy chain disease antibody, and a variable heavy chain immunoglobulin.

96. The method of claim 91, wherein the human patient is optionally administered an immunosuppressant.

97. A method of treating lung transplant rejection in a human patient, the method comprising administering to the patient an effective amount of an antibody that specifically binds tumor necrosis factor alpha.

98. The method of claim 97, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

99. The method of claim 97, wherein the antibody is administered by the route selected from the group consisting of intramuscularly, intravenously, intradermally, cutaneously, subcutaneously, rectally, ionophoretically, topically, locally, and inhalation.

100. The method of claim 97, wherein the antibody is selected from the group consisting of a polyclonal antibody, a monoclonal antibody, a synthetic antibody, a heavy chain antibody and a humanized antibody.

101. The heavy chain antibody of claim 100, wherein the heavy chain antibody is selected from the group consisting of a camelid antibody, a heavy chain disease antibody, and a variable heavy chain immunoglobulin.

102. The method of claim 97, wherein the human patient is optionally administered an immunosuppressant.

103. A method of treating lung transplant rejection in a human patient, the method comprising administering to the patient an effective amount of an antibody that specifically binds interferon gamma.

104. The method of claim 103, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

105. The method of claim 103, wherein the antibody is administered by the route selected from the group consisting of intramuscularly, intravenously, intradermally, cutaneously, subcutaneously, rectally, ionophoretically, topically, locally, and inhalation.

106. The method of claim 103, wherein the antibody is selected from the group consisting of a polyclonal antibody, a monoclonal antibody, a synthetic antibody, a heavy chain antibody and a humanized antibody.

107. The heavy chain antibody of claim 106, wherein the heavy chain antibody is selected from the group consisting of a camelid antibody, a heavy chain disease antibody, and a variable heavy chain immunoglobulin.

108. The method of claim 103, wherein the human patient is optionally administered an immunosuppressant.

109. A method of treating bone marrow transplant rejection in a human patient, the method comprising administering to the patient an effective amount of a combination of an antibody that specifically binds tumor necrosis factor alpha and an antibody that specifically binds interferon, gamma.

110. The method of claim 109, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

111. The method of claim 109, wherein the antibody is administered by, the route selected from the group consisting of intramuscularly, intravenously, intradermally, cutaneously, subcutaneously, rectally, ionophoretically, topically, locally, and inhalation.

112. The method of claim 109, wherein the antibody is selected from the group consisting of a polyclonal antibody, a monoclonal antibody, a synthetic antibody, a heavy chain antibody and a humanized antibody.

113. The heavy chain antibody of claim 112, wherein the heavy chain antibody is selected from the group consisting of a camelid antibody, a heavy chain disease antibody, and a variable heavy chain immunoglobulin.

114. The method of claim 109, wherein the human patient is optionally administered an immunosuppressant.

115. A method of treating bone marrow transplant rejection in a human patient, the method comprising administering to the patient an effective amount of an antibody that specifically binds tumor necrosis factor alpha.

116. The method of claim 15, wherein the antibody is selected, from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

117. The method of claim 115, wherein the antibody is administered by the route selected from the group consisting of intramuscularly, intravenously, intradermally, cutaneously, subcutaneously, rectally, ionophoretically, topically, locally, and inhalation.

118. The method of claim 115, wherein the antibody is selected from the group consisting of a polyclonal antibody, a monoclonal antibody, a synthetic antibody, a heavy chain antibody and a humanized antibody.

119. The heavy chain antibody of claim 118, wherein the heavy chain antibody is selected from the group consisting of a camelid antibody, a heavy chain disease antibody, and a variable heavy chain immunoglobulin.

120. The method of claim 115, wherein the human patient is optionally administered an immunosuppressant.

121. A method of treating bone marrow transplant rejection in a human patient, the method comprising administering to the patient an effective amount of an antibody that specifically binds interferon gamma.

122. The method of claim 121, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

123. The method of claim 121 wherein the antibody is administered by the route selected from the group consisting of intramuscularly, intravenously, intradermally, cutaneously, subcutaneously, rectally, ionophoretically, topically, locally, and inhalation.

124. The method of claim 121, wherein the antibody is selected from the group consisting of a polyclonal antibody, a monoclonal antibody, a synthetic antibody, a heavy chain antibody and a humanized antibody.

125. The heavy chain antibody of claim 124, wherein the heavy chain antibody is selected from the group consisting of a camelid antibody, a heavy,chain disease antibody, and a variable heavy chain immunoglobulin.

126. The method of claim 121, wherein the human patient is optionally administered an immunosuppressant.
